(19) United States
(12) Patent Application Publication
(10) Pub. No.: US 2026/0294534 A1
(43) Pub. Date: Oct. 1, 2026

(54) MEDICAL LASER APPARATUS

(71) Applicant: Optheras A/S, Birkerød (DK)

(72) Inventors: Anders Sig OLESEN, Birkerød (DK); Mark DENNINGER, Birkerød (DK); Thomas SØRENSEN, Birkerød (DK); Per PETERSEN, Birkerød (DK); Felix FLEISCHHAUER, Birkerød (DE); Zahra ALTURFI, Birkerød (DK); Thomas ALKESKJOLD, Birkerød (DK)

(21) Appl. No.: 19/478,752

(22) PCT Filed: Apr. 23, 2024
(86) PCT No.: PCT/EP2024/060982

§ 371 Date: Oct. 24, 2025

(30) Foreign Application Priority Data
Apr. 24, 2023 (EP) 23169493.6

(51) Int. Cl. A61B 18/22; A61B 18/00

(52) U.S. Cl. CPC A61B 18/22; A61B 2018/00517; A61B 2018/00577; A61B 2018/00589; A61B 2018/00648; A61B 2018/00726; A61B 2018/00791; A61B 2018/00982

(57) ABSTRACT

Various aspects disclosed herein relate to a medical laser apparatus, the apparatus comprising: at least one treatment laser source configured to output treatment laser radiation for treatment of a medical condition; one or more optical ports configured for coupling the apparatus to a fiber-optic device, the fiber-optic device including at least one optical fiber and having a proximal end and a distal end, the distal end being configured to be advanced through a working channel of an endoscope towards a treatment site of a subject to be treated, the one or more optical ports comprising an optical output port for outputting the treatment laser radiation; at least one sensor configured to acquire at least one sensor signal indicative of one or more predetermined safety indicators, the one or more safety indicator including at least: a connection indicator indicative of whether a fiber-optic device is coupled to the optical output port, and one or more fiber tip position indicators indicative of whether the distal end of the optical fiber of the coupled fiber-optic device is positioned at a safe location; a control module configured to receive the at least one sensor signal from the at least one sensor and to enable output of the treatment laser radiation via the optical output port only when the at least one sensor signals indicate that a fiber-optic device is coupled to the optical output port and that said distal end is positioned at a safe location.

### TECHNICAL FIELD

The present disclosure relates to a medical laser apparatus for providing laser treatment via minimally invasive procedures using an endoscope.

### BACKGROUND

Many medical conditions can be treated by applying laser light to tissue to be treated. Often, such treatment is performed by directing treatment laser radiation via an optical fiber towards the tissue to be treated, such as to cause ablation and/or coagulation of/in the tissue to be treated.

In many procedures, an optical fiber is advanced through the working channel (often also referred to as lumen) of an endoscope, in particular a flexible endoscope. There are a variety of types of endoscopes configured for viewing and/or treating various parts of the body, such as of the gastrointestinal tract, the respiratory tract or the urinary tract. Endoscopes exist that are specifically configured for reaching particular areas of the body. Examples of such endoscopes include cystoscopes, colonoscopes, rhinoscopes, etc. For example, laser ablation and/or coagulation therapy of bladder cancer is carried out using a cystoscope. A cystoscope is an endoscope that is configured for performing a cystoscopy where the cystoscope is advanced through the urethra into the urinary bladder. To this end, a cystoscope includes a very thin, flexible tube. During laser ablation and/or coagulation therapy of bladder cancer, a healthcare provider inserts a fiber-optic device through a working channel of the cystoscope and into the patient's bladder. The fiber-optic device is then used to target the laser towards the specific area of tissue that needs to be treated.

It is therefore generally desirable to provide a medical laser apparatus that allows an efficient, safe and reliable treatment of a subject. In some embodiments, it is particularly desirable to provide a medical laser apparatus that allows an efficient, safe and reliable treatment of a urinary tract, in particular of the urinary bladder, of a bladder cancer.

In particular, laser therapy, such as ablation and/or coagulation therapy, typically requires powerful laser radiation to be effective. Operation of a medical laser apparatus capable of producing such powerful radiation thus involves the risk of the operator of the apparatus and/or the patient to be unintentionally exposed to potentially hazardous laser radiation. Therefore operation of such a medical laser apparatus often involves personal protection measures for protecting the laser operator and/or the patient, such as protective goggles, door switches, procedural safety measures, etc. Examples of such protection measures include eye protection measures. It is desirable to reduce, or even eliminate, the need for such personal protection measures while still ensuring safe operation of the medical laser apparatus. In particular, it is desirable to ensure operation of the apparatus with no or at least an acceptably low risk of unintentionally exposing the operator's or patient's eyes (or other sensitive body parts that are not to be treated) to hazardous laser radiation.

It is generally desirable to provide a medical laser apparatus that is easy to operate.

Despite previous efforts it remains desirable to provide a medical laser apparatus that solves one or more of the above problems and/or other problems, and/or that has other benefits, or that at least provides an alternative to existing solutions.

### SUMMARY

On this background, various aspects disclosed herein relate to a medical laser apparatus, the apparatus comprising:
at least one treatment laser source configured to output treatment laser radiation for treatment of a medical condition,
one or more optical ports configured for coupling the apparatus to a fiber-optic device, the fiber-optic device including at least one optical fiber and having a proximal end and a distal end, the distal end being configured to be advanced through a working channel of an endoscope towards a treatment site of a subject to be treated, the one or more optical ports comprising an optical output port for outputting the treatment laser radiation,
a control module configured to receive at least one sensor signal indicative of one or more predetermined safety indicators, the one or more safety indicators including at least: a connection indicator indicative of whether a fiber-optic device is coupled to the optical output port, and one or more fiber tip position indicators indicative of whether the distal end of the optical fiber of the coupled fiber-optic device is positioned at a safe location, wherein the control module is further configured to enable output of the treatment laser radiation via the optical output port only when the at least one sensor signal indicates that a fiber-optic device is coupled to the optical output port and that said distal end is positioned at a safe location.

In some embodiments, the one or more safety indicators further include one or more fiber integrity indicators. The one or more fiber integrity indicators may be indicative of sufficient integrity of the coupled fiber-optic device, e.g. that the optical fiber is not broken and/or not bent below a predetermined bending radius. Sufficient integrity refers to an acceptable degree of integrity, e.g. an acceptable degree of optical loss, an acceptable bending radius and/or the like, e.g. as defined by one or more predetermined criteria. The one or more fiber integrity indicators may be indicative of a structural and/or operational integrity of the coupled fiber-optic device, i.e. whether the coupled fiber-optic device is structurally intact and/or operationally intact. At least one of the one or more fiber integrity indicators may be indicative of detected damage of the fiber of the coupled fiber-optic device, e.g. indicative of a broken fiber. At least one of the one or more fiber integrity indicators may be indicative of a detected optical loss in the fiber and/or in another part of the optical path between the treatment laser source and the distal end of the optical fiber, and/or one or more other indicators indicative of whether the optical fiber and/or another portion of the optical path between the treatment laser source and the distal end of the optical fiber is operating as expected. Accordingly, the control module may further be configured to enable output of the treatment laser radiation via the optical output port only when the at least one sensor signals indicate that fiber integrity is intact. Fiber integrity may e.g. be detected by measuring reflected light from the end facet of the fiber tip through one or more optical paths.

In some embodiments, the medical laser apparatus, or a system including the medical laser apparatus, is configured for treatment of a urinary tract, in particular of the urinary bladder, of a subject. Accordingly, the endoscope may be a cystoscope. In other embodiments, the apparatus may be configured for treatment of other conditions, such as conditions in another part of a subject's body.

In some embodiments, the apparatus comprises at least one sensor configured to acquire at least one of the at least one sensor signal indicative of one or more predetermined safety indicators, i.e. the control module may be configured to receive the at least one of the at least one sensor signals from one or more sensors of the apparatus. Alternatively or additionally, the control module may receive at least one of the at least one sensor signals from one or more sensors external to the medical laser apparatus. Accordingly, the control module ay receive the at least one sensor signal from one or more internal and/or external sensors, i.e. from one or more sensors that are comprised in the apparatus and/or from one or more sensors that are external to the apparatus.

The at least one sensor may include an optical sensor of the medical laser apparatus, which optical sensor may be configured to receive and detect radiation from at least one of the one or more optical ports. The detected light is thus received at the distal end of the optical fiber and coupled into the at least one of the optical ports from the proximal end of the optical fiber. The optical sensor may be a passive sensor, i.e. configured to detect light not originating from (or otherwise caused by) the optical sensor itself, or an active sensor, i.e. configured to detect light originating from (or otherwise caused by) the optical sensor itself. A passive optical sensor may e.g. be configured to detect ambient light or light originating from another light source, such as an illumination light of the endoscope.

In some embodiments, the optical sensor is configured to detect light emitted from the endoscope tip, e.g. from a cystoscope tip, being received at the distal end of the optical fiber. In some embodiments, the endoscope light (e.g. endoscope LED) may be configured to emit light at a predetermined modulation (e.g. a pulsed or amplitude-modulated light with a modulation/pulse frequency high enough not to be visible, or a frequency-modulated light) or light having otherwise an encoded signal/signature embedded in it. The optical sensor may detect the endoscope light by detecting light having the predetermined modulation. In other embodiments, the optical sensor may be configured to detect another attribute, e.g. a spectral attribute of the endoscope light.

In some embodiments, the medical laser apparatus includes an active optical sensor that comprises a sensor radiation source configured to emit sensor radiation, and an optical sensor to detect return radiation, such as reflected sensor radiation that has been reflected by a surface (e.g. by an end facet of the optical fiber, by the tissue to be treated, or by another surface onto which the sensor laser radiation impinges) or other return radiation returned by a material onto which the sensor radiation impinges. To this end, the apparatus is configured to output the sensor radiation via at least one of the one or more optical ports and to receive the return radiation via at least one of the one or more optical ports. It will be appreciated that the apparatus may include more than one optical ports, e.g. the optical output port for outputting the treatment laser radiation, one or more optical input ports and/or one or more additional optical output ports. In some embodiments one or more of the optical ports may be operable as both input and output ports. The one or more optical ports may all be coupled to the same fiber-optic device and, in particular, into the same optical fiber. The sensor radiation is thus coupled into the proximal end of the optical fiber and emitted by the distal end of the optical fiber. Similarly, the return light is received at the distal end of the optical fiber and coupled back into at least one of the optical ports from the proximal end of the optical fiber. The sensor radiation source may be a sensor laser source configured to emit sensor laser radiation or another light source for emitting sensor light. Preferably, the sensor radiation is non-hazardous radiation, e.g. sensor laser radiation having an output power sufficiently low to not be hazardous for a human eye, in particular sensor laser radiation that can be classified as laser class 1 according to IEC 60825-1:07-2015. Examples of active optical sensors include an interferometric sensor, such as a broadband interferometric sensor, also referred to herein as an OCT sensor. Other examples include a spectrometric sensor. In other embodiments, the fiber-optic device may include multiple fibers and/or the apparatus may be configured for being coupled to more than one fiber-optic device, e.g. via respective ones of the optical ports.

The optical sensor may detect when the distal end of the optical fiber is inserted into a liquid and/or into the urinary tract and/or into the endoscope. The type of liquid may depend on the type of medical treatment performed. In particular, the liquid may be water, an aqueous solution, or another liquid having water as a major constituent, e.g. a saline solution, urine and/or the like. In some embodiments, the optical sensor of the apparatus includes a Superluminescent Light Emitting Diode (SLED) for emitting an SLED source signal. The optical sensor may thus use the SLED source signal to detect, via a single- or multi-mode fiber path, presence of a liquid, such as water, saline and/or urine, at the fiber facet (i.e. at the distal end of the optical fiber).

The optical sensor may detect the bladder wall or other treatment site/biological tissue to be treated.

The one or more fiber-tip position indicators may include one or more of the following indicators:
a) an insertion indicator indicative of the optical fiber having been inserted into a liquid, such as water, saline and/or urine, and/or the optical fiber having been inserted into the endoscope,
b) an endoscope tip indicator indicative of the optical fiber being positioned at a distal end of a working channel of an endoscope, e.g. as indicated by detecting that light emitted from the endoscope tip, e.g. from a cystoscope tip, is received at the distal end of the optical fiber, and/or as indicated by detecting that light emitted by the distal end of the optical fiber is received at a camera of the endoscope, e.g. by a camera chip located at the endoscope tip receiving;
c) a treatment site indicator indicative of the distal end of the optical fiber being in proximity of a potential treatment site, e.g. as indicated by the optical sensor having detected a potential treatment site, e.g. biological tissue, in particular tissue inside the patient's body, such as the bladder wall, in sufficient proximity between of the distal end of the optical fiber. Sufficient proximity may be defined by the detection range of the laser sensor or by a predetermined threshold distance.

Here and in the following the term potential treatment site does not necessarily imply that the site at which treatment is intended to be performed has to be detected. This term merely implies a site at which treatment is safe to be performed from a laser operation point of view, i.e. without (or at least without unacceptable) risk of the operator of the apparatus being exposed to hazardous radiation or of the patient's eyes or skin being unintentionally exposed to such radiation. For example, detection of a bladder tumor to be treated may not necessarily be required as long as bladder wall tissue in general is detected. Conversely, the term potential treatment site is not intended to be limited to a site that has not yet been treated either; it may also refer to a treatment site where treatment has already begun or may even already have been finished.

In some embodiments, the control module may be configured to only enable the treatment laser radiation to be output when the connection indicator and two or more, such as three or more, fiber tip position indicators are fulfilled, e.g. including one or more of the fiber tip position indicators a)-c) discussed above and/or one or more alternative or additional fiber tip position indicators.

In some embodiments, the control module may be configured to only enable the treatment laser radiation to be output when the connection indicator, the fiber integrity indicator and one, such as two, such as three or more fiber tip position indicators are fulfilled, e.g. including one, such as two, such as three of the fiber tip position indicators a)-c) discussed above and/or alternative or additional fiber tip position indicators.

In some embodiments, the one or more fiber tip position indicators include: an insertion indicator indicative of the optical fiber having been inserted into a liquid, and a treatment site indicator indicative of the distal end of the optical fiber being in proximity of a potential treatment site, and the control module is configured to only enable the treatment laser radiation to be output when at least the insertion indicator and the treatment site indicator are fulfilled.

In some embodiments, the control module may be configured to only enable the treatment laser radiation to be output when at least two of the connection indicator, the fiber integrity indicator and one or more fiber tip position indicators are detected by two separate sensors, respectively, and/or using two separate optical pathways, thus increasing the fault-tolerance of the system.

In some embodiments, the control module may be configured to enable the treatment laser radiation to be output only when at least one safety indicator—selected from the connection indicator, the fiber integrity indicator and the fiber tip position indicators—is detected by two separate sensors, preferably independently, and/or using two separate optical and/or signal processing pathways, thus increasing the fault-tolerance of the system. For example, one or more conditions may be detected by two separate sensors using two separate optical pathways, where one pathway is a single-mode/few-mode pathway with V<10, and the other pathway is a multi-mode pathway with V>10.

For example an interferometric (OCT) system may be used for detecting proximity to tissue and/or detection of immersion of the fiber tip into a liquid, such water, saline and/or urine.

Generally interferometric distance measurements refer to a distance measurement by measuring optical interference between a reference reflection and reflection from a target surface/tissue, the distance to which is to be determined. The reference reflection can e.g. be the reflection from the distal fiber tip (thus facilitating calibration or even avoiding the need for calibration).

A spectrometric measurement may be used for detecting light from the cystoscope/endoscope and/or for detection of immersion of the fiber tip into a liquid.

In some embodiments, the apparatus is configured to process sensor signals from at least one sensor, in particular from at least one optical sensor, using two separate signal processing pathways/pipelines, to obtain two separate safety indicator values of at least one of said safety indicators. In particular sensor data from one or from each sensor may be processed (in the electrical domain) using two parallel and partially or completely separate computational “arms” and/or using two different computational units and/or two different computational algorithms (software algorithms). The control module may be configured to only enable the treatment laser radiation to be output when the processing, when the separate processing pipelines yield the same result, in particular when the two separate safety indicator values are equal or at least only differ from each other within a predetermined margin.

In some embodiments, the control module may be configured to only enable the treatment laser radiation to be output when at least two, such as at least three, such as at least four of the above safety indicators are fulfilled in a predetermined temporal sequence, e.g. only when the connection indicator is fulfilled before the above-mentioned fiber-tip position indicator a), which in turn is fulfilled before fiber tip indicator b) and/or c).

In some embodiments, the apparatus comprises a user input interface, such as a button, touch screen, and/or the like configured to receive a user confirmation that the fiber-optic device is positioned with its distal end inside a patient's body (or at another safe location) and that the apparatus is ready for operation. The user confirmation may include a confirmation that all prescribed procedural safety measures have been successfully performed, e.g. certain checks have been performed. The control module may be configured to only enable the treatment laser radiation to be output when said user confirmation has been received, thereby providing an additional layer of safety.

In some embodiments the control module is configured to keep the output path of the treatment laser radiation blocked by a shutter (“non-contact mode” or “safety mode”) while conducting safety measurements, e.g. including interferometric distance measurements (additional laser safety feature). This may optionally be coupled/connected to a general laser safety interlock, e.g. a laser safety interlock that enables output of the treatment laser radiation only when a fiber-optic device is coupled to the optical output port. In some embodiments, the absence of a coupled fiber-optic device may also be detected by the optical sensor, such as by the laser sensor.

In some embodiments the one or more sensors are configured to perform a time-of-flight measurement between the fiber input (at the proximal end of the optical fiber) and the fiber output facet (at the distal end of the optical fiber); the timing difference may e.g. be about 30 ns for return signal in a 3 m fiber. The time-of-flight measurement may serve as a fiber integrity indicator. Additionally or alternatively, a detected amplitude of reflected pulses from the distal end will change, depending on the fiber tip being in air or a liquid, thereby serving as an indicator as to whether the distal end of the fiber is immersed in the liquid, such as in water, saline, urine or the like. Accordingly, in some embodiments, the apparatus includes a time-of-flight sensor operable to emit light pulses through the connected optical fiber, e.g. through a cladding of a multi-clad fiber, and to detect corresponding return pulses reflected by the fiber output facet. A decrease in amplitude of the detected return pulses from an elevated to a lower level indicates immersion of the optical fiber into water, saline or urine. Similarly, a permanently low amplitude level of the return pulses may indicate that the optical fiber is damaged, not properly connected or that the integrity of the optical path is otherwise compromised.

The apparatus may use a non-optical detection method for detecting insertion of the optical fiber into the endoscope and/or into a liquid, such as water, saline and/or urine, e.g. using a pair of electrical wires brought along side of the fiber buffer.

The detection of the target site and/or a distance between the target site and the fiber tip may be based on interferometric (OCT) detection of biological tissue to verify presence of tissue.

The apparatus may include an optical sensor that is configured to detect ambient light entering the distal end of the optical fiber, e.g. to perform a distinction between light/dark. This may be used to detect whether the fiber tip is inside the working channel of the endoscope. The control module may thus be configured to always turn the treatment laser OFF when in the dark (e.g. as defined by a suitable light threshold) as this may indicate that the fiber tip is inside the working channel or that the endoscope light is OFF. The endoscopic/cystoscopic light may be detected based on detection of a light level and/or spectral properties (e.g. detect LED light or Xenon light) typical or even characteristic of endoscopic/cystoscopic light and/or based on a specifically amplitude or frequency modulated or otherwise specifically encoded endoscopic/cystoscopic light.

In some embodiments, the medical laser apparatus may receive a sensor signal from an external sensor and derive one or more of the safety indicators at least in part based on the received sensor signal from the external sensor. Examples of such an external sensor include a sensor of the endoscope into which the optical fiber is advanced. Examples of a sensor of the endoscope include a camera of the endoscope, i.e. the received sensor signal may include or be derived from the camera signal from such camera. For example, the apparatus may derive a fiber tip insertion indicator from a sensor signal received from a sensor of the endoscope. In particular, according some embodiments, the apparatus further comprises a light source for emitting light via at least one of the one or more optical ports, the emitted light being detectable by a camera of the endoscope, and the control module is configured to receive a camera signal from the camera, to process the received camera signal so as to detect whether the camera has captured said emitted light, and to enable output of the treatment laser radiation via the optical output port only when the control module has detected that the camera has captured said emitted light.

In some embodiments, the apparatus is configured to monitor, while output of the treatment laser radiation is enabled, whether one or more of the safety conditions discussed above remain fulfilled, in particular one or more, such as all, of the connection indicator, the fiber tip position indicator(s) and the fiber integrity indicator. For example, the apparatus may be configured to automatically disable output of the treatment laser radiation responsive to at least one of the conditions no longer being fulfilled, e.g.
If the fiber-optic device is disconnected from the one or more optical ports, in particular from the optical output port
If the optical fiber is pulled back into, or even out of the working channel of the endoscope
If no liquid, such as water, saline and/or urine, is detected
If a distance to the target site exceeds a safety threshold or if no biological tissue is detected
If a reduction of fiber integrity is detected, e.g. a reduction below a predetermined/an acceptable threshold.

In some embodiments, the treatment laser source is configured to receive a heartbeat signal and to stop output of the treatment laser radiation responsive to a failure to receive the heartbeat signal.

The heartbeat signal may be indicative of normal operation of one or more components of the apparatus, in particular of the control module and/or of the at least one sensor. The treatment laser source may be configured to receive the heartbeat signal from the control module and/or directly or indirectly from the at least one sensor.

In some embodiments, the control module may be configured to send a heartbeat signal to the treatment laser source. The control module may be configured to monitor operation of at least some components of the apparatus. For example, the control module may be configured to monitor operation of the at least one sensor, e.g. based on the received sensor signals and/or based on respective sensor heartbeat signals received by the control module. Failure to receive a heartbeat signal from the control module may thus indicate faulty operation of the control module and/or of the monitored aspects of the operation of the apparatus. In some embodiments, the control module may be configured to only emit the heartbeat signal when and as long as the safety indicators are fulfilled.

In some embodiments, the at least one sensor may send a sensor heartbeat signal, e.g. to the control module and/or directly to the treatment laser source. The treatment laser source may be configured to receive the sensor heartbeat signal instead of or in addition to the heartbeat signal from the control module. Accordingly, the control module and/or the treatment laser source may be configured to disable output of the treatment laser radiation if the sensor heartbeat signal is not received, or not received for a predetermined period of time.

The heartbeat signal may be an intermittent signal, in particular a periodic signal emitted at a suitable heartbeat rate, e.g. at a heartbeat rate of between 10 Hz and 100 Hz, or at another suitable update rate. For example, in some embodiments a heartbeat signal may be emitted between every 10 ms and every 20 ms.

The treatment laser source may be configured to emit pulsed treatment laser radiation. In particular, the treatment laser source may be configured to only emit a predetermined number of pulses, e.g. a single pulse or a pulse train of a predetermined length, and to stop emitting further pulses unless a heartbeat signal has been received, within a predetermined period of time. It will be appreciated that the heartbeat rate may be selected in accordance with the predetermined number of pulses and/or the duty cycle of the pulse train. For example, when the treatment laser source is configured to only emit a single pulse of 10 ms, a heartbeat rate of between one heartbeat every 10 ms and every 20 ms may be a suitable choice. It will be appreciated that other embodiments may employ other heartbeat rates and/or other pulse durations.

The treatment laser source may thus comprise a heartbeat monitoring circuit configured to stop the treatment laser source upon failure to receive the heartbeat signal. Preferably the heartbeat monitoring circuit is implemented in hardware, e.g. as a hardware timer which is reset by the heartbeat signal. In some embodiments, the treatment laser source may be configured to only output treatment laser radiation subject to other conditions being fulfilled, such as subject to other signals being received, e.g. subject to receipt of an activation signal indicative of the operator having pressed a foot pedal or other activated the laser treatment.

The control module may be configured to monitor receipt of acceptable signals from the at least one sensor and send its heartbeat signal only subject to receipt of acceptable signals. For example, responsive to the safety indicators being fulfilled and the operator having initiated the treatment, the control module may, optionally subject to receiving the sensor heartbeat signals, control the treatment laser source to emit a preprogrammed pulse sequence. Only subject to the safety features still being fulfilled, optionally only subject to the operator still activating the laser and, optionally, the sensor heartbeat signals still being received, the control module may continue to send its heartbeat signal to cause the treatment laser source to keep emitting another pulse sequence emit a subsequent pulse sequence. The treatment laser source may be configured, preferably implemented in hardware only, such as a hardware timer, to automatically switch the treatment laser source off after a preprogrammed sequence. The treatment laser source may further be configured to fire again if a heartbeat signal is received from the control module.

In some embodiments, the apparatus comprises the treatment laser source and the fiber-optic device.

The apparatus may be configured to optically couple the treatment laser radiation into the fiber-optic device, in particular at or near the proximal end of the fiber-optic device. The apparatus may be configured to emit the treatment laser radiation from the distal end towards tissue at a treatment site to be treated, e.g. tissue of the urinary tract, in particular of the urinary bladder, to be treated.

The treatment laser radiation may have a wavelength suitable for the treatment to be performed. In some embodiments, the wavelength is between 750 nm and 1000 nm, such as between 950 nm and 1000 nm or between 750 nm and 950 nm, such as between 780 nm and 820 nm or between 900 nm and 950 nm, such as between 905 nm and 925 nm. Other useful wavelengths include a wavelength between 1000 nm and 2000 nm, such as between 1400 nm and 2150 nm. Suitable treatment laser sources include diode lasers, Erbium lasers, Thulium lasers, Holmium lasers and/or others. Wavelengths where water has high absorption and for which the fiber-optic device has a high transmittance may be useful. When treatment laser radiation at such a wavelength is emitted toward the tissue to be treated, the water or aqueous solution between the fiber tip and the tissue to be treated is heated up resulting in the creation of bubbles that impinge on the tissue to be treated and facilitate ablated tissue to be removed. Wavelengths where water has a relatively low absorption and the tissue to be treated has a relatively high absorption may also be useful. In particular lipids have a high absorption in the wavelength range between 900 nm and 950 nm, and also the absorption and hemoglobin is relatively high. Accordingly, using a wavelength at these wavelength ranges allows efficient ablation and/or coagulation of tissue by emitting the laser radiation from a fiber tip positioned inside the urinary tract, in particular inside the urinary bladder, at a distance from the tissue, thereby allowing simultaneous illumination of a relative large area or tissue without attenuating the laser radiation due to the passage through the surrounding water or aqueous solution. Moreover, wavelengths outside the visible range may be useful, as these do not negatively affect visual inspection by e.g. a camera, in particular when the wavelength range is between 900 nm and 950 nm. It will be appreciated that emission of treatment laser radiation at multiple wavelengths may also be used, e.g. concurrently or alternatingly. Treatment laser radiation may be applied in longer or short bursts, or otherwise, e.g. applied over longer periods, e.g. for a period during which a user command is received, e.g. a period during which a foot pedal is activated.

In some embodiments, the apparatus is configured to emit the treatment laser radiation at an output power of at least 10 mW, such as at least 50 mW, such as at least 100 mW, such as at least 200 mW, such as at least 500 mW, such as at least 1 W. In some embodiments, the apparatus may even be configured to emit the treatment laser radiation at higher output power, e.g. at bursts between 10 W and 200 W.

Generally, unless specified otherwise, references to numerical values of the power of laser radiation discussed herein, are intended to refer to the peak power of the laser radiation. For example, when the laser radiation is pulsed, the numerical values correspond to the peak power of the individual pulses rather than on the average power of a pulse train. The latter may be smaller than the peak power and related to the peak power by a factor indicative of the duty cycle of the pulse train.

In some embodiments, the apparatus comprises more than one treatment laser source each configured to output respective treatment laser radiation for treatment of said medical condition, e.g. of the urinary tract, in particular the urinary bladder, and to optically couple the respective treatment laser radiation into the fiber-optic device via at least one of the optical ports, in particular via the optical output port. The treatment laser sources may create treatment laser radiation at respective wavelengths different from each other.

For example, selective and alternating application of respective treatment laser radiation may be performed responsive to receipt of corresponding control input by the operator. Accordingly, the operator may control the apparatus to selectively emit different types of treatment laser radiation.

In some embodiments, the apparatus further comprises a pilot light source configured to output visible pilot light and to optically couple the pilot light into the fiber-optic device, wherein the apparatus is configured to emit a pilot beam of said pilot light from the distal end towards tissue to be treated so as to illuminate a target spot on the tissue to be treated by visible pilot light. In particular, in some embodiments, the visible pilot light is multicolored light, which may include two or more color components detectable by a camera of the medical laser apparatus, e.g. including at least two detectable color components that are at least 40 nm apart. The at least two color components may be part of a broad-band emission spectrum having a bandwidth of 40 nm or more, where the intensity of the emitted pilot light may be substantially uniform across the bandwidth or vary across the bandwidth. The two detectable color components may each have an intensity high enough for them to be detectable by a camera or similar detector of the medical laser apparatus such that the spot illuminated by the pilot beam is visible to the human observer in a camera image as an illuminated spot having a color resulting as a mix of said at least two color components, e.g. as a white or substantially white spot. Preferably the pilot beam is configured to illuminate at least the target spot illuminated by the treatment laser radiation. The pilot beam may be emitted before and, optionally, during application of the treatment laser radiation. The pilot beam may be fed through the same optical fiber as the treatment laser radiation or via a separate optical fiber. The inventor has realized that a multi-colored pilot beam provides an improved visibility and is less tiring for the operator when using the apparatus over extended periods of time. However, other types of pilot beams are also contemplated.

In some embodiments, the apparatus comprises a medical treatment device and the fiber-optic device. The medical treatment device may comprise a housing and at least the treatment laser source, accommodated in said housing. The fiber-optic device may be a disposable fiber-optic device configured to be detachably and optically coupled to the medical treatment device, in particular to one or more optical ports of the medical treatment device, more particularly to the optical output port of the medical treatment device. Accordingly, the operational costs are reduced, since only the relatively inexpensive fiber-optic device, which is inserted into the patient's body during the treatment, is disposed after use.

In some embodiments, the optical fiber is an at least double-clad optical fiber and the apparatus is configured to output the treatment laser radiation via a cladding of the at least doubled-clad optical fiber. Accordingly, the apparatus allows multiple types of radiation to be fed through the fiber-optic device without unduly interfering with each other. For example, the apparatus may include an interferometric distance sensor employing a single-mode core of the at least double-clad optical fiber, while the relatively high power treatment laser radiation may be fed through a multi-mode cladding of the at least double-clad optical fiber.

In particular, in some embodiments, the apparatus comprises an optical side combiner configured to couple the treatment laser radiation into said at least one cladding of the at least double-clad optical fiber. Coupling the treatment laser radiation into the at least one cladding of the at least double-clad optical fiber by means of a side combiner facilitates the illumination of a target spot by the treatment laser radiation with an intensity distribution having a peripheral region of higher intensity and a central region of lower intensity. This in turn facilitates a more uniform heating of the tissue to be treated across the entire target spot.

In some embodiments, the apparatus is configured to emit the treatment laser radiation from the distal end as a divergent first treatment beam configured to illuminate a target spot on the tissue to be treated. In particular, in some embodiments, the medical laser apparatus is configured to illuminate a target spot of spot diameter between 1 mm and 10 mm, such as between 1 mm and 7 mm, when the distal end is displaced from the tissue to be treated by between 1 mm and 10 mm, such as between 2 mm and 5 mm. Accordingly, a relatively large target spot may be illuminated and concurrently treated while providing the operator with a better overall view of the target site.

In some embodiments, the tissue to be treated is soft tissue, in particular cancerous soft tissue.

The present disclosure relates to different aspects, including the medical laser apparatus discussed above and further discussed in the following. It will be appreciated that each of the aspects may have one or more embodiments corresponding to the embodiments described in connection with one or more of the other aspects and/or disclosed in the appended claims.

In particular, according to one aspect, disclosed herein are embodiments of a method of operating a medical laser apparatus, e.g. an apparatus for treating cancer of the urinary tract, in particular the urinary bladder, of a subject by laser ablation and/or coagulation.

According to yet another aspect, disclosed herein are embodiments of a method of treating cancer of the urinary tract, in particular the urinary bladder, of a subject by laser ablation and/or coagulation.

According to yet another aspect, disclosed herein are embodiments of a medical laser apparatus, the apparatus comprising:
at least one treatment laser source configured to output treatment laser radiation for treatment of a medical condition,
one or more optical ports configured for coupling the apparatus to a fiber-optic device, the fiber-optic device including at least one optical fiber and having a proximal end and a distal end, the distal end being configured to be advanced through a working channel of an endoscope towards a treatment site of a subject to be treated, the one or more optical ports comprising an optical output port for outputting the treatment laser radiation,
a light source for emitting light via at least one of the one or more optical ports, the emitted light being detectable by a camera of the endoscope;
a control module configured to receive a camera signal from the camera, to process the received camera signal so as to detect whether the camera has captured said emitted light, and to enable output of the treatment laser radiation via the optical output port only when the control module has detected that the camera has captured said emitted light.

According to yet another aspect, disclosed herein are embodiments of a medical laser apparatus, the apparatus comprising:
at least one treatment laser source configured to output treatment laser radiation for treatment of a medical condition,
one or more optical ports configured for coupling the apparatus to a fiber-optic device, the fiber-optic device including at least one optical fiber and having a proximal end and a distal end, the distal end being configured to be advanced through a working channel of an endoscope towards a treatment site of a subject to be treated, the one or more optical ports comprising an optical output port for outputting the treatment laser radiation,
   wherein the treatment laser source is configured to receive a heartbeat signal and to stop output of the treatment laser radiation responsive to a failure to receive the heartbeat signal.

According to yet another aspect, disclosed herein are embodiments of a system comprising:
a medical laser apparatus as disclosed herein, and
an endoscope, in particular a cystoscope, the endoscope having a working channel for receiving the optical fiber, the endoscope further comprising a camera for capturing one or more images of a target site located in front of a distal end of the endoscope.

Those skilled in the art will recognize still other aspects of the present application upon reading and understanding the attached description.

### BRIEF DESCRIPTION OF THE DRAWINGS

The above and other aspects are illustrated by way of example and not limited by the figures of the accompanying drawings, in which like references indicate similar elements and in which:

FIG. 1 diagrammatically illustrates an example of a medical laser apparatus in accordance with embodiments disclosed herein;

FIG. 2 diagrammatically illustrates operation of an example of a medical laser apparatus with its optical fiber positioned at a distance from a target site to be treated in accordance with embodiments disclosed herein;

FIG. 3 diagrammatically illustrates a further example of a medical laser apparatus in accordance with embodiments disclosed herein;

FIG. 4 diagrammatically illustrates an example of a disposable fiber-optic device with its optical fiber having been inserted into and advanced through the working channel of an endoscope in accordance with embodiments disclosed herein;

FIG. 5 diagrammatically illustrates an example of a disposable fiber-optic device in accordance with embodiments disclosed herein;

FIG. 6 diagrammatically illustrates an example of an optical combiner module of a connector module of a disposable fiber-optic device in accordance with embodiments disclosed herein;

FIG. 7 diagrammatically illustrates another example of an optical combiner module of a connector module of a disposable fiber-optic device in accordance with embodiments disclosed herein, wherein the at least double-clad optical fiber includes a tapered region;

FIG. 8 diagrammatically illustrates a more detailed example of a medical laser apparatus in accordance with embodiments disclosed herein;

FIG. 9 diagrammatically illustrates operation of an example of a medical laser apparatus in accordance with various embodiments disclosed herein;

FIGS. 10 and 11 diagrammatically illustrate respective examples of a medical laser apparatus in accordance with embodiments disclosed herein;

FIG. 12 diagrammatically illustrates another example of a medical laser apparatus in accordance with embodiments disclosed herein;

FIG. 13 diagrammatically illustrates another example of a medical laser apparatus in accordance with embodiments disclosed herein.

### DETAILED DESCRIPTION

The following describes embodiments of a medical laser apparatus that mitigate one or more of the above-noted and/or other shortcomings of existing devices or that can at least serve as an alternative to existing devices.

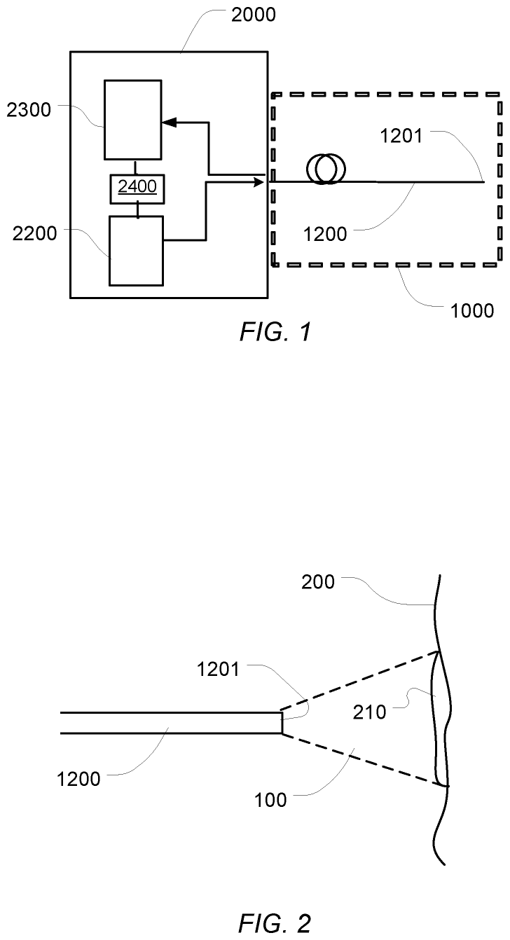

FIG. 1 diagrammatically illustrates an example of a medical laser apparatus in accordance with embodiments disclosed herein.

Various embodiments of the medical laser apparatus are for use in a clinical setting, in particular operated by hospital/clinical/medical staff.

The medical laser apparatus comprises a medical treatment device 2000 and a fiber-optic device 1000. The fiber-optic device 1000 may be configured to be detachably and optically coupled to the medical treatment device 2000; in particular, the fiber-optic device 1000 may be disposable, e.g. after single-use. The fiber-optic device may be coupled to the medical treatment device via one or more suitable connectors.

The fiber-optic device 1000 comprises an optical fiber 1200, e.g. a multi-mode fiber or a double- or multi-clad optical fiber.

The optical fiber 1200 may have a diameter suitable for being inserted into the working channel of a cystoscope or other type of endoscope, i.e. the choice of diameter may depend on the type of endoscope. Suitable diameters may be between 0.1 mm and 1 mm, such as between 0.1 mm and 0.4 mm, preferably between 0.1 mm and 0.3 mm, such as between 0.1 mm and 0.25 mm. Optical fibers having a diameter of 1 mm or less, preferably 0.4 mm or less, such as 0.3 mm or less are sufficiently thin to allow bending with relatively little induced tensile and compressive stress on the fiber edges. The length of the at least double-clad optical fiber may depend on the type of endoscope. Suitable lengths may be between 2 m and 4 m.

Generally, at least in some embodiments, the optical fiber, which emits the radiation towards the tissue to be treated, is an at least double-clad optical fiber. It may have a fiber diameter between 100 μm and 1000 μm, such as between 100 μm and 300 μm, such as between 150 μm and 200 μm. In some embodiments the numerical aperture of the multi-mode cladding, which conveys the treatment laser radiation has a numerical aperture of between 0.17 and 0.50, such as between 0.22 and 0.45. Here, the numerical aperture of the inner cladding may be defined as the square root of the difference between the refractive index squared of the inner cladding and the refractive index squared of the outer cladding, which surrounds the inner cladding.

In some embodiments, the distal end 1201 of the optical fiber 1200 may be angled, preferably with rounded edges for easy insertion into the working channel of the endoscope and reduced risk of edges of the optical fiber breaking of during insertion.

Generally, the medical treatment device 2000 includes a treatment laser source 2200 configured to output treatment laser radiation for treatment of a medical condition. In some embodiments, the treatment laser radiation is for ablation and/or coagulation treatment of a bladder cancer or another medical condition of the urinary tract, in particular treatment laser radiation suitable for treating tumor tissue of the urinary tract, in particular the urinary bladder, by ablation and/or coagulation. The treatment laser source 2200 may include a suitable type of laser, e.g. a diode laser, configured to emit treatment laser radiation at a wavelength and power suitable for the medical treatment to be performed. When the medical laser apparatus is for treatment of tumors of the urinary tract, in particular the urinary bladder, by ablation and/or coagulation, the treatment laser source may comprise a high-brightness diode laser. The treatment laser radiation may be optically coupled into the optical fiber 1200, e.g. at or near the proximal end of the optical fiber. The optical fiber is preferably configured to receive the treatment laser radiation, in particular a treatment laser radiation having an output power of at least 10 mW and a suitable wavelength, e.g. as discussed in the summary section above.

In the present embodiment, the medical treatment device 2000 further includes a sensor module 2300. The sensor module may be configured to receive light entering the distal end 1201 of the optical fiber 1200, and include one or more optical sensors for detecting the received light.

In some embodiments, the sensor module 2300 may include a sensor laser source configured to output sensor laser radiation. The medical laser apparatus may be configured to output the sensor laser radiation via the optical fiber. To this end, the optical fiber 1200 may be an at least double-clad optical fiber, and the medical laser apparatus may be configured to output the sensor laser radiation via a core of the at least doubled-clad optical fiber 1200. The at least one core of the at least double-clad optical fiber may have a V number of less than 10, preferably less than 8, such as less than 6, such as less than 3 or even 2.4, at a wavelength of the sensor laser radiation, i.e. the at least one core may be a few-mode core or even a single-mode core at a wavelength of the sensor laser radiation.

In some embodiments, the sensor laser radiation may include multiple wavelengths. Accordingly, the V number of the least one core of the at least double-clad optical fiber may be different for the different wavelengths of the sensor radiation. For example the core may be a single-mode core at one or some wavelengths of the sensor laser radiation and a few-mode core (or even a multi-mode core) at other wavelengths of the laser radiation. In particular, in some embodiments, the medical laser apparatus includes more than one sensor module, e.g. an interferometric distance sensor module and a spectroscopic sensor module. The different sensor modules may operate at different wavelengths. In such embodiments, the at least one core of the at least double-clad optical fiber may be a few-mode core with a small V number or even a single-mode core (e.g. having a V number of less than 4, such as less than 3 or even 2.4) at a wavelength of the sensor laser radiation of one of the sensor modules, e.g. of the interferometric sensor module. The at least one core of the at least double-clad optical fiber may be a few-mode core having a larger V number (e.g. between 3 and 10, such as between 3 and 6) at a wavelength of the sensor laser radiation of the other sensor module, e.g. of the spectroscopic sensor module.

In various embodiments, the medical treatment device 2000 comprises an interferometric distance sensor module configured to perform interferometric distance sensing of the distance between the distal end of the optical fiber 1200 and the tissue to be treated. In particular, in some embodiments, the interferometric distance sensor is a common path sensor where the distal end of the optical fiber is operable as a reflector of a reference optical path, thus providing a particularly compact system, which does not require any additional fibers. The medical treatment device is preferably further configured to control the treatment laser radiation at least in part responsive to the measured distance.

In some embodiments, the sensor module is configured to detect light emitted by an endoscope, in particular at a distal end of the endoscope, and reflected from tissue or other material positioned in front of the distal end of the endoscope. The endoscope light may thus be received by the distal end of the optical fiber and be detected by the sensor module 2300.

The fiber-optic device is configured to be inserted into the working channel of an endoscope, such as of a cystoscope, e.g. as described in connection with FIGS. 2 and/or 4. The endoscope/cystoscope may be a conventional endoscope/cystoscope or an endoscope/cystoscope specifically adapted for use with the medical laser apparatus disclosed herein.

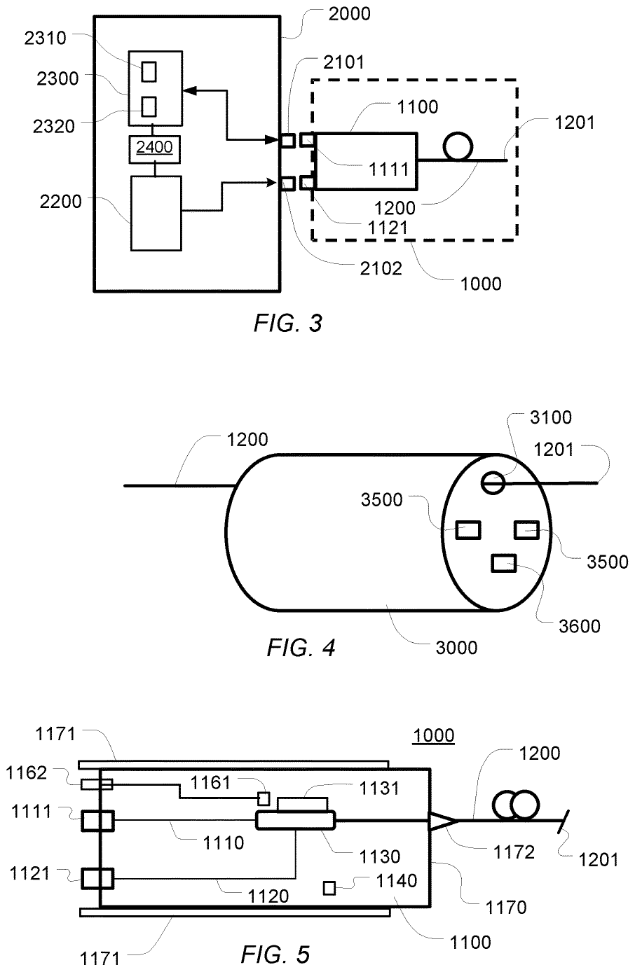

The medical treatment device 2000 may provide a single fiber connector for coupling a fiber to the medical treatment device that conveys the treatment laser radiation and the sensor radiation, e.g. as described in connection with FIG. 11. In other embodiments, the medical treatment device 2000 may provide two (or more) separate fiber connectors for coupling respective fibers to the medical treatment device that may then be combined into a single fiber in a separate combiner module, e.g. as described in connection with FIGS. 3, 5-8 and/or 10.

The medical treatment device may comprise a control module 2400 for controlling operation of the treatment laser source and or other components of the medical treatment device, e.g. a shutter, the sensor module 2300 or the like. To this end the control module may receive sensor signals or data based on the sensor signals from the sensor module 2300. Generally, the control module may be implemented as an MCU or in another suitable manner. The control module may be implemented as a single circuit or as multiple devices or circuits that may perform respective aspects or parts of the control operations.

FIG. 2 diagrammatically illustrates operation of an example of a medical laser apparatus with its optical fiber positioned at a distance from bladder tissue or other treatment site to be treated in accordance with embodiments disclosed herein.

Generally, various embodiments of the medical laser apparatus is for treatment of the urinary tract, e.g. for treatment of the urinary bladder, in particular for treatment of bladder cancer. In use, the distal end 1201 of the optical fiber 1200 is inserted into the working channel of a cystoscope adapted for insertion into the urinary tract, in particular the urinary bladder, through the urethra. Working channels of cystoscopes typically have a diameter of between 1.5 mm and 2 mm, such as 1.8 mm. A cystoscope typically has to be able to be bent with a small bending radius, e.g. with a bending radius of 10 mm or less, or even 5 mm or less. When inserted into the urinary tract, in particular the urinary bladder, the distal end 1201 of the optical fiber 1200 is brought into an operational position at a distance from the tissue 210 to be treated, which may be a tumor of the urinary bladder 200. The apparatus emits treatment laser radiation from the distal end 210 of the optical fiber 1200 towards the tissue to be treated so as to cause ablation and/or coagulation of the tissue 210.

The treatment laser radiation may be directed to the target tissue in a number of ways. For example, when the optical fiber 1200 emits the treatment laser radiation as a divergent beam 100, a relatively large target spot can be illuminated and, hence, treated at a time, thus reducing the need to move the fiber around so as to illuminate different locations while providing a uniform treatment of a larger target spot.

In some embodiments, during emission of the first the distance between the distal end of the optical fiber 1200 and the tissue 210 to be treated is between 2 mm and 6 mm, such as between 3 mm and 5 mm.

It will be appreciated that the medical laser apparatus of any of the embodiments described herein may include a pilot light source, e.g. a pilot laser source or other type of light source adapted to emit visible light suitable as pilot beam to illuminate the portion of the tissue to be treated and to serve as aiming guide to the physician or other user of the apparatus. The pilot beam may be fed through the same optical fiber as the treatment laser radiation or through a separate optical fiber. In some embodiments, the pilot beam may be multi-colored. In some embodiments, the apparatus may be configured to control a visible attribute, e.g. a color and/or intensity, of the pilot light responsive to one or more operational parameters, user inputs and or sensor signals.

FIG. 3 diagrammatically illustrates yet another example of a medical laser apparatus in accordance with embodiments disclosed herein.

The apparatus of FIG. 3 is similar to the apparatus of FIG. 1 in that it comprises a medical treatment device 2000 and a fiber-optic device 1000. The fiber-optic device 1000 includes an optical fiber 1200 and the medical treatment device includes a treatment laser source 2200, a control module 2400, and a sensor module 2300, all as described in connection with FIG. 1.

The disposable fiber-optic device 1000 is configured to be detachably and optically coupled to the medical treatment device. To this end, the disposable fiber-optic device 1000 of this embodiment comprises an optical fiber 1200. The disposable fiber-optic device 1000 may further comprise a connector module 1100 for coupling the optical fiber 1200 to the medical treatment device 2000.

In this embodiment, the optical fiber 1200 is at least double-clad, i.e. it may be double-clad or it may be multi-clad with more than two claddings, e.g. triple-clad. Generally, at double-clad optical fiber comprises three layers of optical material. The inner-most layer is called the core. It is surrounded by an inner cladding, which is surrounded by an outer cladding. The three layers are typically made of materials with different refractive indices. Other embodiments may use other types of optical fibers.

The distal end 1201 of the at least double-clad optical fiber 1200 is configured to be advanced through a working channel of an endoscope, such as a cystoscope. Preferably, the core is a single-mode core, single-mode at least at one or more target wavelengths or range of target wavelengths. Optionally, the at least double-clad optical fiber 1200 includes two or more cores, preferably single-mode cores, e.g. a few-mode or single-mode central core and one or more few-mode or single-mode side cores. Optionally, the at least double-clad optical fiber has a medical-grade buffer coating, such as blue EFTE, Teflon, Nylon etc. The optical fiber may have a round inner cladding or a hexagonal or octagonal inner cladding for mode scrambling. The inner cladding may be a multi-core cladding.

The connector module 1100 may be configured for optically connecting the proximal end of the at least double-clad optical fiber 1200 to the medical treatment device 2000. To this end, the connector module 1100 comprises one or more optical connectors. In some embodiments, the connector module 1100 is configured for separately coupling a core and a cladding, respectively, of the at least double-clad optical fiber 1200 to the medical treatment device 2000. To this end, the connector module 1100 may have a first optical connector and a second optical connector. The first optical connector may be configured for detachable connection to a first mating optical connector of the medical treatment device 2000. The second optical connector may be configured for detachable connection to a second mating optical connector of the medical treatment device. An embodiment of the disposable fiber-optic device 1000 will be described in greater detail below with reference to FIGS. 4 and 5.

The medical laser apparatus may be configured to output the treatment laser radiation via a cladding, in particular the inner cladding, of the at least doubled-clad optical fiber 1200. The cladding may be a multi-mode cladding.

In the present embodiment, the sensor module 2300 is configured to output and receive sensor laser radiation through the at least doubled-clad optical fiber 1200 and to optically measure one or more parameters of the treatment based on the received sensor laser radiation. To this end, the sensor module 2300 may include a sensor laser source 2311 configured to output the sensor laser radiation. The medical laser apparatus may be configured to output the sensor laser radiation through a core of the at least doubled-clad optical fiber 1200.

The sensor module 2300 may further comprise a detector 2312 for receipt and detection of radiation received from the treatment site, e.g. from the tissue to be treated, in particular radiation reflected by the target site in response to being illuminated by the sensor laser radiation from the sensor laser source 2311 and/or in response to being illuminated by light emitted from the distal end of the endoscope. The detector 2312 may be configured to receive the radiation from the target site via the at least double-clad optical fiber 1200 of the disposable fiber-optic device. The detector 2312 may be configured to receive the radiation from the target site via the at least one core and/or via a cladding of the at least double-clad optical fiber 1200 of the disposable fiber-optic device.

The medical laser apparatus may be configured to time-division multiplex the output of the treatment laser radiation and at least the optical measurement of the one or more parameters. Accordingly, some embodiments of the medical laser apparatus alternatingly emit the treatment laser radiation and perform optical measurements, i.e. some embodiments of the medical laser apparatus intermittently interrupt emission—or at least to substantially reduce emission—of the treatment laser radiation to perform the optical measurements, thereby allowing reliable optical measurements to be performed during the treatment. In some embodiments, the medical laser apparatus is configured to adjust one or more parameters, in particular the output power, a duty cycle and/or a pulse duration, of the treatment laser radiation responsive to the measured one or more parameters. Accordingly, the medical laser apparatus may be configured to control the treatment laser source to resume emission of the intermittently interrupted treatment laser radiation with the adjusted one or more parameters. Generally, it will be appreciated that the time-division multiplexing may comprise a complete interruption of the emission of the treatment laser radiation to perform the optical measurements or at least a substantial reduction of the emitted treatment laser radiation while performing the optical measurements, e.g. a reduction to no more 5% of the output power of the treatment laser radiation used during the treatment cycle, such as a reduction to no more than 1%.

In some embodiments, the medical laser apparatus is configured to output the treatment laser radiation as pulsed laser radiation, i.e. as a train of treatment laser pulses mutually separated by time intervals between the treatment laser pulses. The medical laser apparatus may be configured to emit the sensor laser radiation during some or all of the time intervals between the treatment laser pulses.

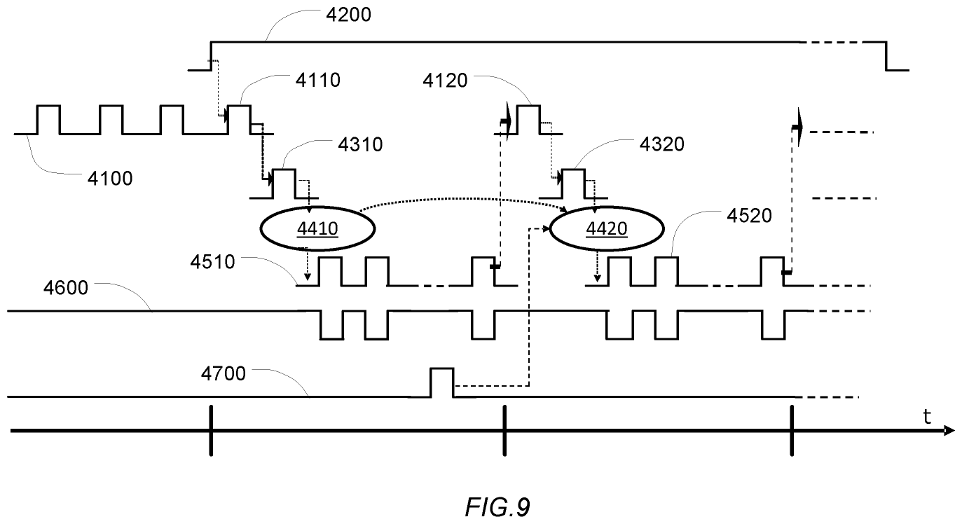

An example of the operation of the medical laser apparatus will be described in more detail below with reference to FIG. 9.

In some embodiments, the sensor laser radiation and the treatment laser radiation have different wavelengths. In particular, the sensor laser radiation may have one or more sensor wavelengths in a first wavelength range and the treatment laser radiation may have one or more treatment wavelengths in a second wavelength range, different and, preferably, spaced apart from the first wavelength range. Accordingly, the treatment laser radiation and some or all of the sensor laser radiation may be spatially separated in the core and cladding of the at least double-clad optical fiber, time-division multiplexed as well as spaced apart in wavelength/frequency.

The sensor laser radiation may have an output power smaller than the treatment laser radiation. For example, the sensor laser radiation may have an output power of less than 5 mW, such as less than 3 mW, such as less than 2 mW, such as less than 1 mW, such as less than 0.5 mW. The sensor laser radiation may be laser radiation that is so weak that no damage can occur to the human eye, in particular such that it can be classified as a class 1 laser according to IEC 60825-1:07-2015. The treatment laser radiation may have an output power of 10 mW or more, as described herein. A beam of the treatment laser radiation may be hazardous to the human eye when viewed by an unprotected observer. The same may apply to specular or diffuse reflections of the treatment laser radiation. However, embodiments of the medical laser apparatus disclosed herein provide protection measures for ensuring that no hazardous emission of radiation can unintentionally be viewed by the person operating the device, the patient, or other person in the vicinity of the apparatus.

The medical treatment device 2000 comprises a control unit 2400 configured to control operation of the medical treatment device 2000. In particular, the control unit may control operation of the treatment laser source 2200 and of the sensor module. In some embodiments, the control unit controls the time-division multiplexing of the laser radiation and the optical measurements based on the sensor laser radiation. The control unit 2400 may be configured to control various other optical treatment and/or sensor modules of the medical treatment device. The control unit may be configured to control the treatment laser source, sensor laser source and/or other radiation sources and/or sensors responsive to received sensor signals. The control unit may include suitable drivers and/or communications interfaces and a processing unit, e.g. a suitable programmed central processing unit. In particular, the control unit may be operable to selectively enable output of the treatment laser radiation only when one or more detectable safety indicators are fulfilled. To this end, the control unit may operate a mechanical shutter and/or other type of shutter, and or selectively allow or prevent the turning ON of the treatment laser source.

In various embodiments, the measured one or more parameters include a distance between the distal end 1201 of the at least double-clad optical fiber 1200 and the tissue to be treated. To this end, in various embodiments, the sensor module 2300 comprises a first optical sensor module, in particular an interferometric distance sensor module, configured to perform interferometric distance sensing of a distance between the distal end 1201 of the at least double-clad optical fiber 1200 and the tissue to be treated. In particular, in some embodiments, the interferometric distance sensor is a common path sensor where the distal end 1201 of the at least double-clad optical fiber is operable as a reflector of a reference optical path, thus providing a particularly compact system, which does not require any additional fibers. The medical treatment device 2000, in particular the control unit 2400 of the medical treatment device, is preferably further configured to control the treatment laser radiation responsive to the measured distance. In particular, the control unit may be configured to control a laser power or another operational parameter of the treatment laser radiation—e.g. a duty cycle of a pulse train of laser cycles, a pulse width or duration of the emission of the laser radiation, or the like—responsive to the sensed distance. For example, the control unit may be configured to control a laser power of the treatment laser radiation responsive to the sensed distance such that the intensity of the laser radiation impinging on the tissue to be treated remains substantially constant despite the varying distance. In another example, the control unit may be configured to selectively enable/disable output of the treatment laser radiation from the medical treatment apparatus responsive to the sensed distance such that the treatment laser radiation is only enabled when the distal end of the optical fiber is in an operational position in front of, in particular in close proximity to, the treatment site. The control unit may further be configured to selectively control a laser power or other operational parameter of the treatment laser radiation responsive to the sensed distance such that the energy of laser radiation impinging on the tissue to be treated during a certain time period remains substantially constant despite the varying distance.

The first optical sensor module may further be configured to detect and distinguish a plurality of different types of material located in front of the distal end of the at least double-clad optical fiber. In such embodiments, the control unit may further be configured to selectively disable/enable emission of the treatment laser radiation and/or control a laser power and/or other operational parameter of the treatment laser radiation responsive to the detected type of material. In particular, the first optical sensor module may be configured to distinguish between biological tissue, such as soft biological tissue, and non-biological material, in particular surfaces of artificial material, such as an interior or exterior surface of the endoscope. Alternatively or additionally, the first optical sensor module may be configured to detect and distinguish whether the distal end of the at least-double clad optical fiber is immersed in a liquid, in particular water or an aqueous solution such as saline or urine, or whether the distal end is immersed in air.

In some embodiments, the sensor module 2300 comprises a second optical sensor module, in particular a spectroscopic sensor module, configured for spectroscopic analysis of the tissue to be treated and to compute an indicator of a progress of the treatment. To this end, the spectroscopic sensor may be configured for sensing one or more spectral properties of radiation reflected by the tissue to be treated. The medical treatment device may further be configured to determine a measure indicative of a status or progress of the treatment from the sensed one or more spectral properties. Preferably, the medical treatment device is configured to control the treatment laser radiation responsive to the determined status or progress, in particular responsive to the sensed distance and the determined status or progress.

The spectroscopic sensor module may comprise a supercontinuum white-light source for illumination through the single-mode core of the at least double-clad optical fiber 1200. Alternatively, the spectroscopic sensor module may comprise a number of WDM multiplexed LEDs for illumination through the single-mode core of the at least double-clad optical fiber 1200. The spectroscopic sensor module may be operable to measure a reflective state of the tissue to be treated, in particular by measuring a wavelength dependent back-reflection, e.g. via the multi-mode inner cladding of the at least double-clad optical fiber 1200. The spectroscopic sensor module may comprise two or more LEDs, that are turned on/off one by one, and the detector may be operable to detect a power of the back-reflected light using a simple photo-detector. The detected power difference between the signals at different wavelengths may be used by the control unit of the medical treatment device to estimate the reflective state of the tissue. The spectroscopic sensor module may measure the Hemoglobin state by measuring the reflected light, for example in the range between 500 nm and 600 nm, where Hb absorbs. The spectroscopic sensor module may emit short wavelength light, e.g. blue light, to excite fluorophores in the tissue, and detect the fluorescence. In any event the control unit may use the signals from the spectroscopic sensor to determine optimal settings of the treatment laser.

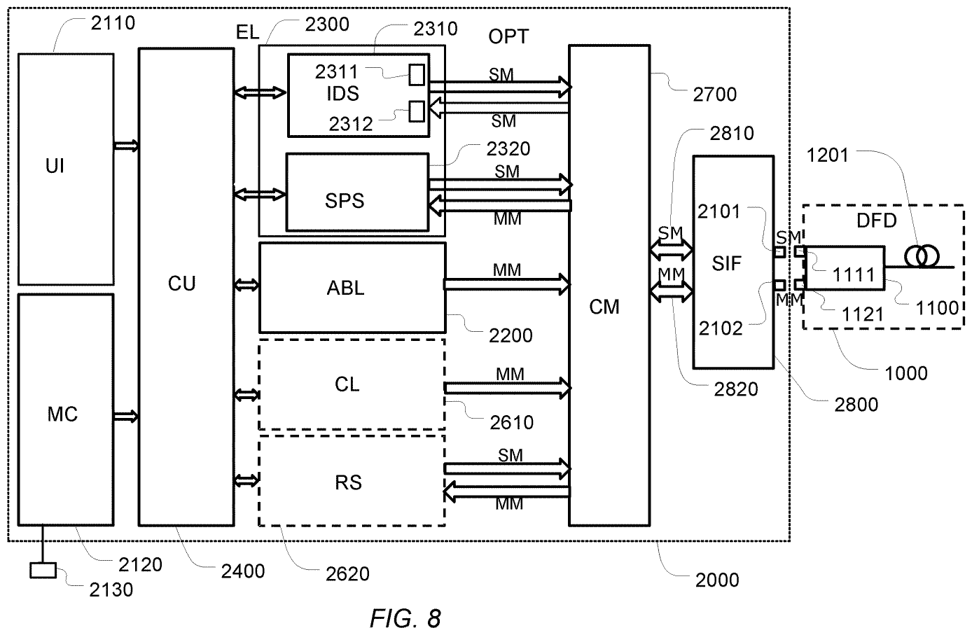

An embodiment of a medical treatment device 2000 will be described in greater detail below with reference to FIG. 8. As will be apparent from the disclosure below, the medical treatment device may include additional or alternative radiation sources and/or additional optical detectors.

In use, the disposable fiber-optic device 1000 is detachably and optically connected to the medical treatment device 2000 via the connector module 1100, and the distal end 1201 of the at least double-clad optical fiber 1200 is inserted into the working channel of an endoscope, e.g. as illustrated in FIG. 4.

FIG. 4 diagrammatically illustrates an example of an optical fiber 1200, e.g. the optical fiber 1200 of any of the embodiments of a medical laser apparatus described herein. FIG. 4 shows the optical fiber 1200 having been inserted and advanced into the working channel 3100 of an endoscope 3000, in particular a cystoscope. In the example of FIG. 4 the distal end 1201 of the optical fiber 1200 extends out of the working channel of the endoscope. In use, the endoscope is inserted into a vessel or organ of a patient, in particular into the urinary bladder via the urethra. The endoscope 3000 may be inserted with the optical fiber 1200 already inserted in the working channel 3100; alternatively, the optical fiber 1200 may be inserted into the working channel 3100 after insertion of the endoscope 3000 into the target organ or vessel.

Endoscopes adapted for various medical procedures are known as such in the art and will not be described in greater detail herein. In particular, different types of endoscopes may have working channels of more or less standardized diameter. Typical working channels of endoscopes have a diameter of between 1 mm and 5 mm. Some endoscopes may further include a camera 3600 and/or one or more illumination lights, e.g. LEDs 3500, arranged at a distal end of the endoscope 3000 so as to allow a physician to observe progress of the medical procedure. In other examples, the illumination light may be fed via an optical fiber to the distal end of the endoscope.

FIG. 5 diagrammatically illustrates an example of a disposable fiber-optic device 1000 in accordance with embodiments disclosed herein.

The disposable fiber-optic device 1000 comprises an at least double-clad optical fiber 1200 having a proximal end and a distal end 1201. The distal end 1201 is configured to be advanced through a working channel of an endoscope, e.g. as described in connection with FIG. 4. The at least double-clad optical fiber 1200 has at least one core, an inner cladding surrounding the core, and at least one outer cladding surrounding the inner cladding. In particular, the at least double-clad optical fiber 1200 may be a double-clad optical fiber having one or more cores, an inner cladding and an outer cladding.

The disposable fiber-optic device 1000 further comprises a connector module 1100 for optically connecting the proximal end of the at least double-clad optical fiber 1200 to a medical treatment device (not shown in FIG. 5), e.g. as described in connection with FIG. 3.

In the embodiment of FIG. 5, the connector module 1100 is configured for separately coupling a core and an inner cladding, respectively, of the at least double-clad optical fiber 1200 to the medical treatment device. The connector module 1100 comprises a first optical fiber 1110, a second optical fiber 1120, a first optical connector 1111 for detachably and optically connecting the first optical fiber 1110 to medical treatment device, a second optical connector 1121 for detachably and optically connecting the second optical fiber 1120 to the medical treatment device, and an optical combiner module 1300 configured to couple radiation between the first optical fiber 1110 and the at least one core of the at least double-clad optical fiber 1200 and to couple radiation between the second optical fiber 1120 and at least one cladding of the at least double-clad optical fiber 1200.

The first optical connector 1111 is configured for detachable connection to a first mating optical connector of the medical treatment device and to optically couple the core of the at least double-clad optical fiber to the first mating optical connector. The second optical connector 1121 is configured for detachable connection to a second mating optical connector of the medical treatment device and to optically couple the inner cladding of the at least double-clad optical fiber to the second mating optical connector.

In some embodiments, the treatment laser source of the medical treatment device is optically coupled via the second mating optical connector to the second optical connector 1121 of the disposable fiber-optic device 1000. Accordingly, the second optical connector is preferably configured to receive treatment laser beam, in particular a treatment laser beam having an output power of more than 10 mW.

The sensor laser module of the medical treatment device may optically be coupled via a first mating optical connector to the first optical connector 1111 of the disposable fiber-optic device. Accordingly, the first optical connector is preferably configured to receive a sensor laser beam and to output reflected laser radiation.

Generally, detachable connection refers to a connection during normal use performed by a user of the medical laser apparatus where the connection can be disconnected again during normal use. In particular, detachable connection of an optical connector of the disposable fiber-optic device implies that the corresponding mating connector of the medical treatment device, to which the optical connector is connected, is not destroyed during the connection or disconnection. Preferably, the optical connector of the disposable fiber-optic device is not destroyed during the disconnection either, i.e. the optical connectors of the disposable fiber-optic device can reversibly and repeatedly be connected to the medical treatment device. Optical connection implies that radiation can pass between the medical treatment device and the corresponding optical fiber of the connector module to which the optical connector is coupled.

Generally, in some embodiments, the connector module may comprise a housing 1170. The first optical fiber 1110, the second optical fiber 1120 and the optical combiner module 1300 may completely or partly be accommodated within the housing. The housing 1170 may be a plastic housing or a housing made from another suitable material, e.g. a rigid material or a soft material. The housing may be a box, e.g. made from a rigid material, or it may be provided in another form, e.g. as an elongated flexible sleeve. The housing may form a single compartments or multiple compartments. The housing may have one end configured to be connected to the medical treatment device to thereby connect the first and second optical connectors to corresponding first and second mating optical connectors of the medical treatment device. To this end, the first and/or second optical connector(s), which may be spring-loaded, may be mounted directly on or in a side wall of the housing, thus allowing easy and reliable connection. Alternatively, the first and/or second optical connector(s), which may be spring-loaded, may be mounted to the housing via one or more flexible fiber-optic cables. Accordingly, in general, the components of the connector module may all be accommodated in a single housing or they may distributed between two or more separate housings, which may be interconnected via one or more fiber-optic cables or otherwise.

Generally, the first and/or second optical connector may be spring-loaded. To this end, the first and/or second optical connector may include a spring or other elastic element for holding the first or second optical connector, respectively, in position when connected to the medical treatment device. The spring or other elastic element may be any suitable component for imparting an elastic force, e.g. a flat spring, a coil spring, etc.

The housing 1170 may comprise additional mechanical connectors or guides 1171, e.g. guiderails, that further facilitate a reliable and safe connection, e.g. by ensuring fixation to the medical treatment device with a suitable mechanical force on the spring-loaded connectors. To this end, the guiderails or other mechanical connectors may include a click-on mechanism.

The housing 1170 further includes an opening through which the at least double-clad optical fiber 1200 protrudes out of the housing. The housing 1170 may include a soft cone 1172 or other form of stress and/or bending relief at the opening through which the at least double-clad optical fiber 1200 protrudes out of the housing.

The first optical fiber 1110 may be a single-mode optical fiber or an optical fiber (e.g. a double-clad optical fiber) having a single-mode core or it may include a single-mode optical fiber section. The first optical fiber, or at least a core of the first optical fiber, may be single-mode at least at a predetermined target wavelength, in particular the wavelength used by a sensor module of the medical treatment device, e.g. by an interferometric distance sensor module. The first optical fiber 1110 may be a single fiber section or include multiple fiber sections optically coupled to each other, e.g. spliced or otherwise connected to each other.

The first optical connector 1111 may be a low-reflectivity optical single-mode fiber connector, such as a connector providing physical contact between respective fiber ends of the optical fibers that are optically connected by the optical connector, i.e. the first optical fiber 1110 and a corresponding optical fiber of the medical treatment device. In particular, the first optical connector may be an Angled Physical Contact (APC) Connector. In some embodiments the low-reflectivity optical single-mode fiber connector has a reflectivity of less than −40 dB, such as −50 dB, preferably less than −60 dB, such as less than −70 dB, such as −80 dB. Generally, the first optical connector may be configured to couple few-mode or even single-mode laser radiation, in particular radiation characterizable by a V number smaller than 10, such as smaller than 3, into the first optical fiber. The first optical connector may be configured to couple single-mode laser radiation, having a power of less than 1 W, such as less than 200 mW, such as less than 100 mW, such as less than 10 mW, such as less than 5 mW, such as less than 2 mW into the first optical fiber.

Accordingly, an efficient optical coupling between single-mode or few-mode optical fibers with little back-reflection is provided. This is particularly advantageous when the radiation through the core of the at least double-clad optical fiber 1200 is laser radiation for sensor applications, in particular for interferometric measurements such as interferometric distance measurements.

Generally, the terms single-mode radiation or single-mode laser radiation as used herein refer to laser radiation that is transmitted through emitted from a single-mode optical fiber or other single-mode waveguide. Similarly, the terms multi-mode radiation or multi-mode laser radiation as used herein refer to laser radiation that is transmitted through and/or emitted from a multi-mode optical fiber or other multi-mode waveguide.

The second optical fiber 1120 may be a multi-mode optical fiber or include a multi-mode fiber section. The second optical fiber 1120 may be a single fiber section or include multiple fiber sections optically coupled to each other, e.g. spliced or otherwise connected to each other.

The second optical connector 1121 may be a high-power optical multi-mode connector such as an SMA905 connector or a collimated beam connector. A collimated beam connector is a fiber-optic connector including a collimating lens operable to collimate a beam exiting the fiber-optic connector. In particular, the collimating lens may be arranged inside the connector housing of the connector. The collimating lens allows mating of the collimated beam connector with a corresponding collimated beam connector with particularly low power loss. Accordingly, a high-power connection suitable for e.g. high-power treatment laser radiation is provided. The second optical connector may be a free-standing fiber tip connector without physical contact between the fiber ends. Here, the term high-power optical connector is intended to refer to an optical connector adapted for optical connection of optical fibers carrying high-power laser radiation, in particular laser radiation at powers suitable for medical treatment of tissue, e.g. by ablation and/or coagulation. The second optical connector 1121 may be adapted for optical connection of optical fibers carrying laser radiation having a power of 10 mW or more, such as a power of 100 mW or more, such as of 200 mW or more, such as of 1 W or more, such as of 10 W or more, such as 100 W or more, such as 200 W or more, such as between 10 W and 500 W. Generally, the second optical connector may be configured to couple multi-mode laser radiation, in particular radiation characterizable by a V number larger than 10, into the second optical fiber.

Generally, two optical connectors may be operable to be connected with each other via a mating sleeve. Accordingly, in some embodiments, each of the mating optical connectors of the medical treatment device that are operable to be coupled to the optical connectors of the disposable fiber-optic device may comprise or be attached to a respective mating sleeve. The mating sleeve of the second mating optical connector of the medical treatment device that is operable to be coupled to the second optical connector of the disposable fiber-optic device may at least in part be made from a suitable material having a sufficiently high melting point and/or low thermal expansion to allow a high-power optical connection. Examples of suitable materials include Tungsten Carbide.

Generally, the first optical connector 1111 and the second optical connector 1121 provide separate optical coupling of the first optical fiber 1110 and the second optical fiber 1120, respectively, with the medical treatment device. In particular, the first optical connector 1111 provides optical coupling of the first optical fiber 1110 with a corresponding first fiber of the medical treatment device, and the second optical connector 1121 provides optical coupling of the second optical fiber 1120 with a corresponding second fiber of the medical treatment device, the corresponding second fiber of the medical treatment device being different from the corresponding first fiber of the medical treatment device.

In some embodiments, the first and second optical connectors are physically separate connectors. In particular, the optical connector 1111 may have a first connector housing, e.g. including a first ferrule, and the second optical connector 1121 may have a second connector housing, e.g. including a second ferrule, different from the first connector housing. In other embodiments, the first optical connector 1111 and the second optical connector 1121 are accommodated in a single connector housing, or they may otherwise be formed as a single combined connector having multiple optical termini. For example, the single connector housing may be a two-ferrule connector housing, such as a Diamond DM4 connector, having multiple optical termini.

The at least double-clad optical fiber 1200 may feed single-mode, low power sensor laser radiation through its core towards the tissue to be treated. The at least double-clad optical fiber 1200 may further feed single-mode reflected radiation through its core from the distal end of the at least double-clad optical fiber via the first optical connector 1111 to the medical treatment device. The reflected radiation may be sensor laser radiation having been reflected by the distal end 1201 of the at least double-clad optical fiber 1200 and/or sensor laser radiation having been reflected by the tissue to be treated and captured by the distal end 1201 of the at least double-clad optical fiber 1200. Accordingly, the core of the at least double-clad optical fiber 1200 is preferably a single-mode core at least at the wavelength or wavelengths of the sensor laser radiation. The at least double-clad optical fiber 1200 may further feed high-power multi-mode treatment laser radiation through its inner cladding towards the tissue to be treated.

For the purpose of the present description, the term “tissue to be treated” is intended to not only refer to the tissue prior to being treated but also to the tissue during the treatment, including to the tissue having already been affected by the treatment. Generally, the tissue to be treated is biological tissue, such as soft tissue.

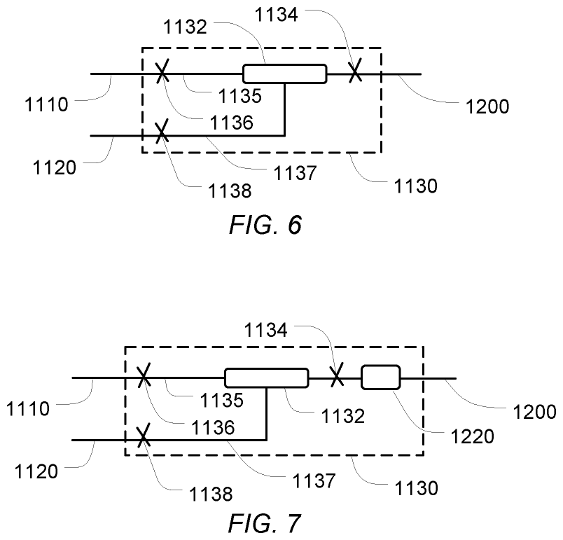

Examples of the optical combiner module 1130 will be described in greater detail with reference to FIGS. 6-7.

Optionally, the connector module 1100 may comprise additional components.

In particular, in some embodiments, the connector module comprises a temperature sensor 1161, e.g. a thermistor, configured to sense the temperature of the optical combiner module 1130. The temperature sensor may be a PTC or NTC sensor. The temperature sensor 1161 may be electrically connectable via electrical connector 1162 to the medical treatment device, thus allowing the medical treatment device to monitor the temperature of the optical combiner module during operation and, optionally, to control operation of the treatment laser source responsive to the sensed temperature. For example, the medical treatment device may be controlled to reduce the power and/or duty cycle of the treatment laser, or even turn the treatment laser off or close a shutter/optical switch, so as to prevent overheating of the optical combiner module 1130.

Alternatively or additionally, the connector module may comprise a cooling member 1131 configured for passive or active cooling of the optical combiner module 1130. The cooling member may e.g. comprise a heat sink, cooling ribs and/or a thermal conductor configured to transport heat into a heat sink of the medical treatment device when the connector module is connected to the medical treatment device.

Yet alternatively or additionally, the disposable fiber-optic device 1000, in particular the connector module 1100, may include an RFID chip 1140 or other form of device-identifier, e.g. a wirelessly readable ID tag, allowing the medical treatment device to automatically register an identifier of the disposable fiber-optic device 1000. This may be advantageous when the medical treatment device is configured to be operated with different types of disposable fiber-optic devices. It allows the medical treatment device to adapt one or more of its operational parameters to the specific type of disposable fiber-optic device being connected.

FIG. 6 diagrammatically illustrates an example of an optical combiner module 1130 of a connector module of a disposable fiber-optic device in accordance with embodiments disclosed herein.

The optical combiner module 1130 couples radiation between the first optical fiber 1110 of the connector module and a core of the at least double-clad optical fiber 1200 of the disposable fiber-optic device according to an embodiment disclosed herein. The optical combiner module 1130 further couples radiation between the second optical fiber 1120 of the connector module and a cladding, in particular an inner cladding, of the at least double-clad optical fiber 1200.

The first optical fiber 1110 may thus be a single-mode optical fiber, at least at one or more target wavelengths, and the second optical fiber 1120 may be a multi-mode optical fiber.

The optical combiner module 1130 comprises an at least double-clad optical fiber section 1135 having a core and a cladding. The core of the at least double-clad optical fiber section 1135 is a few-mode, preferably a single-mode core at a target wavelength of the radiation received via the first optical fiber. The at least double-clad optical fiber section 1135 may include a low-index acrylate coating, optionally with fluorinated silica (F—SiO2) cladding. The core of the at least double-clad optical fiber section may have a diameter of between 5 m and 10 m and/or a numerical aperture of between 0.07 and 0.15. The core of the at least double-clad optical fiber section has a V number at a wavelength λₛₑₙₛₒᵣ of the sensor laser radiation of V<10 @λₛₑₙₛₒᵣ, preferably V<8 @λₛₑₙₛₒᵣ, such as V<7 @λₛₑₙₛₒᵣ, such as V<2.4 @λₛₑₙₛₒᵣ.

The V number is a dimensionless parameter which is often used in the context of optical fibers, such as step-index fibers. It is a normalized frequency parameter, which determines the number of modes of an optical fiber, such as a step-index fiber. It is defined as

$$V = \frac{2 π}{λ} a NA = \frac{2 π}{λ} a \sqrt{{n}_{core}^{2} - {n}_{cladding}^{2}}$$

where λ is the vacuum wavelength, a is the radius of the fiber core and NA is the numerical aperture. n_{core} and n_{cladding} refer to the refractive index of the core and of the inner cladding, respectively.

For the purpose of the present description, and unless otherwise stated, the terms single-mode fiber and single-mode core refer to a fiber and core, respectively, that has a V number less than 2.4 at the relevant wavelength. The terms few-mode fiber and few-mode core refer to a fiber and core, respectively, that has a V number larger than 2.4 and less than 10 at the relevant wavelength. The terms multi-mode fiber and multi-mode core refer to a fiber and core, respectively, that has a V number larger than 10.

The optical combiner module 1130 further comprises a first splice point 1136, which couples radiation between the first optical fiber 1110 and the core of the at least double-clad optical fiber section 1135. The first splice point 1136 may be provided with a cladding mode stripper configured for removal of return multi-mode radiation into the first optical fiber 1110. Alternatively or additionally, the first splice point 1136 may be provided with a scattering surface or a high index glue and/or coating.

The optical combiner module 1130 further comprises a multi-mode fiber section 1137 and a second splice point 1138, which couples radiation between the second optical fiber 1120 and the multi-mode fiber section 1137. The multi-mode fiber section 1137 may have a core diameter of between 50 μm and 100 μm and/or a numerical aperture of 0.27 or less, e.g. of 0.22 or less. The core of the multi-mode fiber section 1137 may have a V number V>10.

The optical combiner module 1130 further comprises an optical fiber combiner 1132 configured for coupling radiation between the multi-mode fiber section 1137 and the inner cladding of the at least double-clad optical fiber section 1135. The optical fiber combiner 1132 may be a side combiner, sometimes also referred to as a side pump combiner, i.e. a combiner configured to laterally couple radiation between the multi-mode fiber section 1137 and the inner cladding of the at least double-clad optical fiber section 1135 while the core of the at least double-clad optical fiber section passes through the side combiner. Hence, the side combiner couples/fuses a multi-mode secondary fiber (here the multi-mode fiber section 1137) at an angle to the circumferentially outer side of a pass-through primary fiber (here the at least double-clad optical fiber section 1135). Preferably, the secondary fiber has a diameter smaller than the primary fiber. For example, the double-clad feed through fiber may have a diameter of 200 μm (or another suitable diameter) and the secondary fiber may have a fiber diameter of 125 μm (or another suitable diameter smaller than the diameter of the feed-through double-clad fiber). The side coupling may result in an up-conversion of the numerical aperture (NA) of the multi-mode inner cladding of the at least double-clad optical fiber section 1135 and, hence, in the at least double-clad optical fiber 1200, e.g. from NA of 0.22 to about 0.45. This in turn results in a larger spot size of the treatment laser radiation on the tissue to be treated. Moreover, the side combiner allows the sensor laser radiation propagating in the core of the at least double-clad optical fiber section to pass through the side combiner without reflections due to splices or the like.

The optical coupler module 1130 further comprises a third splice point 1134 for coupling the at least double-clad optical fiber section 1135 and the proximal end of the at least double-clad optical fiber 1200, in particular for coupling the cores and claddings of the respective fibers.

FIG. 7 diagrammatically illustrates another example of an optical combiner module 1130 of a connector module of a disposable fiber-optic device in accordance with embodiments disclosed herein. The optical combiner module 1130 is similar to the optical combiner module of FIG. 6 in that it comprises an at least double-clad optical fiber section 1135, a multi-mode fiber section 1137, an optical fiber combiner 1132, a first splice point 1136, a second splice point 1138 and a third splice point 1134, all as described in connection with FIG. 6.

The optical combiner module of FIG. 7 differs from the optical combiner module of FIG. 6 in that it is configured to couple the first and second optical fibers 1110 and 1120, respectively, with a multi-core at least double-clad optical fiber 1200, i.e. the optical combiner module of FIG. 7 is for use with embodiments of a disposable fiber-optic device comprising a multi-core at least double-clad optical fiber.

In some embodiments, one or more of the splice points shown in FIGS. 6 and 7 may be omitted. For example, the at least double-clad optical fiber section 1135 may be formed by the proximal end of the at least double-clad optical fiber 1200. Alternatively or additionally, the multi-mode fiber section 1137 may be a portion of the second optical fiber 1120 attached to the second optical connector.

Again referring to FIG. 7, the optical combiner module 1130 of this embodiment further comprises a tapered fiber region 1220 at the proximal end of the at least double-clad optical fiber 1200. The tapered region 1200 is configured for coupling of radiation between a single core of the at least double-clad optical fiber 1200 and one or more further cores of the at least double-clad optical fiber 1200. The tapered region 1220 may be packaged in a ferrule with air surrounding the taper waist to enable a high numerical aperture so as to support up-converted cladding radiation.

FIG. 8 diagrammatically illustrates a more detailed example of a medical laser apparatus in accordance with embodiments disclosed herein.

The medical laser apparatus comprises a medical treatment device 2000 and a fiber-optic device 1000, all as described in connection with FIGS. 1 and 7.

The example medical laser apparatus is configured for treatment of cancer of the urinary bladder by laser ablation and/or coagulation using a cystoscope. It will be appreciated, however, that other embodiments of the medical laser apparatus may be configured for other types of medical procedures and/or for use with other types of endoscopes.

The medical treatment device 2000 comprises various laser sources, optical detectors and accompanying control circuitry, user interfaces, etc. The medical treatment device 2000 may be embodied with all its modules accommodated in a single housing or with respective modules accommodated in different housings.

The medical treatment device 2000 comprises a user-interface module 2110, e.g. comprising a display, such as a touch-sensitive display and suitable user input devices, such as a keyboard, touch screen etc., allowing an operator to control user-controllable functions of the medical treatment device and to allow the medical treatment device to output information relevant for the treatment. In some embodiments, the user-interface module comprises an audible and/or visible power/progress indicator configured to output an audible and/or visual indication indicative of a current laser power of the treatment laser radiation or a current status of the treatment. For example the power indicator may indicate the output power of the treatment laser or the measured treatment status, such that the physician knows when to move the fiber to the next spot. An audible indicator may indicate the power level or treatment status by the volume or pitch of the audible signal, by a repetition rate of repeated tones, or in another suitable manner.

The medical treatment device 2000 may further comprise one or more control devices 2120, such as a foot pedal, e.g. to allow a physician to activate a treatment laser while manipulating the cystoscope.

The medical treatment device further comprises a control unit 2400 electrically and/or otherwise communicatively connected to the user-interface module 2110 and to the one or more control devices 2120. The control unit 2400 comprises circuitry configured to control various optical treatment and/or sensor modules of the medical treatment device. In particular, the control unit may be configured to control a treatment laser source, a sensor laser source and/or other radiation sources and/or sensors responsive to input received from the user-interface module and/or the one or more control devices. The control unit may further control the treatment laser source, sensor laser source and/or other radiation sources and/or sensors responsive to received sensor signals. The control unit may include suitable drivers and/or communications interfaces and a processing unit, e.g. a suitable programmed central processing unit.

The medical treatment device 2000 comprises a treatment laser source 2200 configured to output treatment laser radiation, in particular radiation suitable for treating tumor tissue of the urinary bladder by ablation and/or coagulation. The treatment laser radiation from the treatment laser source 2200 is preferably fed through the second optical connector 1121 and the cladding of the at least double-clad optical fiber 1200 of the disposable fiber-optic device 1000.

The medical treatment device further comprises a sensor module 2300. The sensor module comprises a first optical sensor module 2310 configured for interferometric distance sensing of the distance between the distal end of the at least double-clad optical fiber 1200 and the tissue to be treated.

Preferably, the medical treatment device 2000 may be configured to control the treatment laser source 2200 responsive to the sensed distance between the distal end of the at least double-clad optical fiber 1200 and the tissue to be treated, e.g. by reducing the output power and/or duty cycle of the treatment laser radiation or even shutting down the treatment laser radiation when the fiber tip gets close to the tissue. This may prevent unintended damage to the tissue to be treated.

Interferometric distance sensing may be performed using broadband interferometry known as such from optical coherence tomography (OCT). To this end, the first optical sensor module may comprise a suitable sensor laser source 2311 configured to output single-mode laser radiation in a suitable wavelength, e.g. selected in the range between 800 nm and 1100 nm. In applications such as cystoscopy, it is preferred that the wavelength of the sensor laser radiation is chosen at low-water absorption wavelengths, such as wavelengths smaller than 900 nm or wavelength at about 1050 nm. The sensor laser radiation may have an output power that is considerably smaller, e.g. by at least 2 or 3 orders of magnitude, than the output power of the treatment laser radiation.

The first optical sensor module 2310 further comprise a spectrometer 2312 to perform broadband interferometry. To this end, the first optical sensor module receives reflected radiation back via the disposable fiber-optic device, in particular reference radiation reflected by the tip of the at least double-clad optical fiber 1200 and radiation reflected by the tissue to be treated and captured by the tip of the at least double-clad optical fiber 1200. Based on the reflected radiation, the medical treatment device determines the distance between the distal end of the at least double-clad optical fiber 1200 and the tissue to be treated in a manner known as such in the art. Accordingly, the first optical sensor module 2310 may output sensor laser radiation via the single-mode or few mode core of the at least double-clad optical fiber and receive reflected radiation via the single-mode or few mode core of the at least double-clad optical fiber as a basis for the distance measurement.

Various embodiments of the interferometric distance sensor of the first optical sensor module 2310 provide an axial resolution of between 5 μm and 200 μm, such as between 10 μm and 150 μm, e.g. between 50 μm and 100 μm.

The interferometric distance sensor of the first optical sensor module 2310 may be implemented in different ways. In some embodiments the interferometric distance sensor employs spectral domain OCT using a SLED and a spectrometer. The inventors have found that such a system provides sufficient axial resolution with a bandwidth of the SLED of about 10 nm-30 nm. Accordingly, the spectrometer only needs to cover a relatively small bandwidth as well. Preferably, the spectrometer has a resolution of between 0.01 nm-0.05 nm, thereby obtaining a sufficient axial range suitable for distance measurements during treatment. The interferometric distance sensor may operate at wavelengths in the range between about 800 nm and about 900 nm where CMOS detectors/spectrometers have good sensitivity and InGaAs or GaAs SLEDs are readily available. This maximum axial range of the distance sensor may be between about 3 mm to 10 mm, such as between about 3 mm to 9 mm.

In alternative embodiments, the interferometric distance sensor may employ spectral domain OCT using a swept-source laser and a detector. This embodiment has the advantage of an extended maximum axial range to between about 10 mm and 20 mm. This embodiment may be implemented similar to conventional swept-source OCT systems, with synchronized data acquisition to the laser sweep. The sweep range may be selected to be between 10 nm and 40 nm, such as about 20 nm or less, and the sweep frequency may be selected to be between 1 kHz and 100 kHz, such as 10 kHz or less.

In order to accommodate measurements via a fiber advanced through a flexible endoscope, which is bent and moved around during treatment, various embodiments of the interferometric distance sensor employ a common path interferometric measurement, where the at least double-clad optical fiber 1200 is used both a reference path and as a sample path. Reflection from the distal end of the at least double clad optical fiber is used as reference signal. Hence, a separate reference arm is avoided, and thereby reducing system complexity and costs.

In such embodiments, a suitable amplitude of the reference signal ensures a good interference signal on the spectrometer. To this end, a suitable reference signal amplitude may be obtained by selecting a suitable fiber tip angle of the distal end of the at least double-clad optical fiber, e.g. an angle between 2 and 10 degrees, depending on the fiber type and laser power.

In various embodiments, the radiation from the sensor laser source 2311 of the interferometric distance sensor 2310 as well as the back-reflected radiation are fed through the single-mode core of the at least double-clad optical fiber 1200 and through the first optical connector 1111 of the disposable fiber-optic device 1000. The inventors have realized that the signal path from the spectrometer of the interferometric distance sensor to the distal end of the at least double-clad optical fiber has low back-reflection losses, preferably less than −60 dB. Accordingly, in some embodiments, the first optical connector 1111 of the disposable fiber-optic device, through which the sensor signals for interferometric distance measurement are fed, is preferably a low-reflection connector, e.g. a fiber optical connector with physical contact between fibers, optionally an angled connector such as an E2000 connector.

In some embodiments, the interferometric distance sensor may also be used to detect whether the connected optical fiber is immersed in liquid such as water, saline or urine. When the connected optical fiber is immersed in such a liquid, the level of the return light reflected by the output facet of the connected optical fiber drops as described herein. This drop is observable over a broad wavelength range and can thus be detected by the interferometric distance sensor as a drop in intensity of the reflected intensity over a broad wavelength range, thereby serving as a reliable indicator of the fiber tip being immersed in water, saline, urine or the like.

The sensor module 2300 may preferably comprise a second optical sensor module 2320 configured for spectroscopic analysis of the tissue to be treated. In particular, spectral properties of radiation reflected by the tissue to be treated may be used as an indicator of the progress of the treatment. To this end, the medical treatment device may be configured to measure tissue state using reflectance spectroscopy, e.g. using white-light reflectance spectroscopy.

As the tissue is photo-treated, it changes state from an absorbent state to a more scattering state. Low wavelength light will be more reflected than long wavelength light, and by measuring the difference in reflected power at one or more wavelengths, the scattering state of the tissue can be estimated. Measuring the difference in power between two or more wavelengths of back-reflected radiation thus yields a measure of the photocoagulation state of the tissue and, hence, of the status or progress of the treatment. Accordingly, in various embodiments, the medical treatment device is configured to monitor the status of coagulation caused by the treatment laser using reflectance spectroscopy.

In some embodiments, the second sensor module 2320 comprises an illumination source using a supercontinuum laser, e.g. covering from a wavelength range between 400 nm and 1100 nm, such as between 400 nm and 800 nm. Alternatively the illumination source may us use one or more single-colored LEDs at respective wavelengths, e.g. at one or more of the following wavelengths: about 400 nm, about 500 nm, about 600 nm and about 700 nm and/or other wavelengths in the range between 400 nm and 700 nm.

In any event, the second optical sensor module 2320 is configured to receive radiation reflected by the tissue to be treated. The second optical sensor module 2320 may thus comprise a suitable detector for measuring the reflected radiation. The detector may e.g. comprise a grating CCD/CMOS spectrometer, e.g. with up to 1 nm resolution, covering a suitable wavelength range, e.g. between 400 nm and 1100 nm, such as between 400 nm and 800 nm. Alternatively, the detector may include a photodetector, which may be time-multiplexed with a plurality of LEDs.

To maintain good signal strength of the radiation reflected back from the tissue, it is preferred that the illumination light passes through the first optical connector 1111 and through the single-mode core of the at least double-clad optical fiber 1200 of the disposable fiber-optic device 1000, i.e. that the illumination light shares the same path through the disposable fiber-optic device as the interferometric distance measurement radiation. Spectroscopy is a non-interferometric method, and hence more limited by noise than an interferometric distance measurement. Therefore, in various embodiments, the multi-mode cladding of the at least double-clad optical fiber 1200 of the disposable fiber-optic device 1000 captures the reflected light of the spectroscopic illumination, i.e. the second optical sensor module 2320 receives radiation reflected back from the tissue to be treated responsive to being illuminated by the illumination source of the second optical sensor module 2320 via the cladding of the at least double-clad optical fiber 1200 and via the second optical connector 1121 of the disposable fiber-optic device 1000.

Accordingly, the medical laser apparatus may display or otherwise output a progress indication or even automatically control the treatment laser source 2200, e.g. to prevent unintended overtreatment which might otherwise result in undesired tissue damage. In some embodiments, the second optical sensor module 2320 may output sensor laser radiation via the single-mode or few mode core of the at least double-clad optical fiber and receive reflected sensor laser radiation via the single-mode or few mode core or via the multi-mode cladding of the at least double-clad optical fiber. The wavelengths used for spectral analysis may preferably be chosen at low-water absorption wavelengths, such as wavelengths smaller than 900 nm or wavelengths at about 1050 nm. In particular, in some embodiments, the wavelengths used for spectral analysis may preferably be chosen such that they correspond with absorption maxima hemoglobin and/or another suitable molecular marker.

In some embodiments, the medical treatment device may include additional or alternative optical components, e.g. one or more of the following further components: Some embodiments of the medical treatment device 2000 comprise at least one additional treatment laser 2610, e.g. configured for cutting tissue rather than for ablation and/or coagulation. The cutting laser may output treatment laser radiation at a suitable wavelength, e.g. infrared radiation, such as at 1940 nm, which is absorbed by water. Treatment laser radiation from the cutting laser 2610 is preferably fed through the second optical connector 1121 and the cladding of the at least double-clad optical fiber 1200 of the disposable fiber-optic device 1000. In some embodiments, the additional treatment laser 2610 outputs treatment laser radiation configured to be absorbed by water in the vicinity of the tissue to be treated and to facilitate removal of treated tissue from the treatment site. To this end, the additional treatment laser 2610 may be configured to output treatment laser radiation at a wavelength between 1000 nm and 2000 nm, such as between 1200 nm and 2000 nm.

Alternatively or additionally, the medical treatment device 2000 may comprise a Raman sensor 2620 or other suitable sensor configured for determining properties of a tumor to be treated. The Raman sensor may e.g. employ a 785 nm 300 mW Raman laser. Sensor laser radiation from the Raman laser is preferably fed through the first optical connector 1111 and the single-mode core of the at least double-clad optical fiber 1200 of the disposable fiber-optic device 1000. Return radiation to the Raman sensor is preferably fed through the cladding of the at least double-clad optical fiber 1200 and the second optical connector 1121 of the disposable fiber-optic device 1000.

Yet alternatively or additionally, the medical treatment device may include a pilot light source, e.g. a pilot laser source or other type of light source adapted to emit visible light suitable as pilot beam to illuminate the portion of the tissue to be treated and to serve as aiming guide to the physician or other user of the apparatus. The pilot beam may be fed through the at least double-clad optical fiber 1200, e.g. through the core and/or the inner cladding of the at least double-clad optical fiber 1200. In some embodiments, the pilot beam may be multi-colored. In particular, the pilot beam may include two or more color components detectable by a camera of the medical laser apparatus, e.g. including at least two detectable color components that are at least 40 nm apart. The at least two color components may be part of a broad-band emission spectrum having a bandwidth of 40 nm or more, where the intensity of the emitted pilot light may be substantially uniform across the bandwidth or vary across the bandwidth. The two detectable color components may each have an intensity high enough for them to be detectable by a camera or similar detector of the medical laser apparatus such that the spot illuminated by the pilot beam is visible to the human observer in a camera image as an illuminated spot having a color resulting as a mix of said at least two color components, e.g. as a white or substantially white spot.

In some embodiments, the medical laser apparatus comprises a handheld communication device 2130. The handheld communication device may be communicatively coupled to the medical treatment device 2000, e.g. via a wired or wireless connection. The handheld communication device comprises a user input device, e.g. a push button, that is configured to receive user input from a patient during treatment with the medical laser apparatus. The user input may be indicative of pain or discomfort experienced by the patient during the treatment. To this end the patient may be instructed to press a button if the patient experiences pain or discomfort during the treatment. In some embodiments, the user input device may be configured to measure a force or pressure applied to the user input. The medical treatment device may thus derive a degree of pain or discomfort from the measured force or pressure. The control unit 2400 of the medical treatment device may thus be configured to select a laser power or a pulse duration or another operational parameter of the treatment laser radiation at least in part responsive to the received user input. To this end, the control unit may compare the information from the user device to a current, calculated or selected operational parameter of the treatment laser radiation and adjust the operational parameter to minimize pain of the patient. For example, the control unit may compare the information from the user device to the current, calculated or selected laser power and adjust a maximum power of the treatment laser radiation to minimize pain of patient.

In some embodiments, the medical treatment device 2000 further comprises an optical combiner module 2700 having optical interfaces to the treatment laser source 2200, the first optical sensor module 2310 with an interferometric distance sensor, and the optional second optical sensor 2320. In embodiments with additional optical treatment and/or sensor units, the optical combiner module also has optical interfaces to any such units, e.g. to a cutting laser 2610 and/or a Raman sensor 2620.

As was described above, the treatment laser source 2200 emits relatively high-power treatment laser radiation to be fed through the multi-mode cladding of the at least double-cad optical fiber while the interferometric distance sensor of the first optical sensor module 2310 emits and receives low-power radiation via the single-mode or few-mode core of the at least double-cad optical fiber. The second optical sensor module 2320 emits radiation to be fed through the single-mode or few.-mode core of the at least double-clad optical fiber 1200 of the disposable fiber-optic device, and receives radiation through the cladding of the at least double-clad optical fiber 1200 of the disposable fiber-optic device.

Accordingly, the optical combiner module 2700 is configured to combine the optical paths fed through the core of the at least double-clad optical fiber 1200 via the first optical connector 1111 of the disposable fiber-optic device 1000 with one another. The optical combiner module 2700 is further configured to combine the optical paths fed through the cladding of the at least double-clad optical fiber 1200 via the second optical connector 1121 of the disposable fiber-optic device 1000. To this end, the optical combiner module 2700 may comprise suitable fused fiber combiners for coupling the signal paths fed through the single-mode core of the at least double-clad optical fiber 1200. Similarly, the optical combiner module may comprise suitable free-space components, lenses and dichroic filters to perform multi-mode power and wavelength splitting/combination of the radiation fed through the cladding of the at least double-clad optical fiber 1200.

The optical combiner module 2700 comprises two optical interfaces to be coupled to the disposable fiber-optic device 1000: a single-mode or few-mode interface 2810 (e.g. characterizable by a V number smaller than 10) and a multi-mode interface 2820 (e.g. characterizable by a V number larger than 20), respectively.

Generally, the optical combiner module may comprise one or more fused fiber couplers configured to multiplex sensor laser radiation from the interferometric distance sensor with sensor laser radiation from the spectroscopic sensor in to an optical fiber that is single-mode at least at a wavelength of the sensor laser radiation of the interferometric distance sensor. In particular, the wavelength(s) of the sensor laser radiation of the interferometric distance sensor may be different from the wavelength(s) of the spectroscopic sensor. The fiber couplers may be fused 3 dB couplers.

In various embodiments, the optical combiner module may be configured to multiplex multi-mode treatment laser radiation and multi-mode sensor radiation into a multi-mode optical fiber. To this end, the optical combiner module may include free space components including one or more lenses and one or more dichroic mirrors. The multi-mode sensor radiation may include multi-mode response radiation, which has been reflected by the tissue to be treated in response to being illuminated by sensor laser radiation emitted by the spectroscopic sensor module.

The medical laser apparatus preferably comprises a sacrificial interface module 2800 having corresponding optical interfaces to interface with the single-mode or few-mode interface 2810 and the multi-mode interface 2820, respectively. The sacrificial interface module 2800 optically connects the single-mode or few-mode interface 2810 with a first mating optical connector 2101 to which the first optical connector 1111 of the disposable fiber-optic device 1000 is connectable. Moreover, the sacrificial interface module 2800 optically connects the multi-mode interface 2820 with a second mating optical connector 2102 to which the second optical connector 1121 of the disposable fiber-optic device 1000 is connectable. During operation of the medical laser apparatus, different disposable fiber-optic devices may interchangeably be connected to the mating first and second optical connectors 2101 and 2102, respectively, of the sacrificial interface module 2800, as the disposable fiber-optic device 1000 is typically a single-use device that is replaced by a new disposable fiber-optic device between each treatment. Therefore, the first and second mating optical connectors of the sacrificial interface may be subject to wear. Accordingly, provision of a sacrificial interface module 2800 that can relatively easily be replaced facilitates maintenance of the medical treatment device 2000.

It will be appreciated that the radiation form/to the respective optical modules of the medical treatment device may not necessarily be fed through the disposable fiber-optic device concurrently. Instead, at least some of the radiation is time-multiplexed in a suitable manner to avoid that they interact with each other in an undesired manner. An example of time-division multiplexed operation will be described in more detail below with reference to FIG. 9.

Various embodiments of operating a medical laser apparatus will now be described. Generally, in various embodiments, the medical laser apparatus comprises a control unit that is configured, responsive to a received user input, to emit the treatment laser radiation from the distal end towards tissue of the urinary tract.

In some embodiments, the medical laser apparatus comprises a control unit that is configured, responsive to a received user input, to
1) activate a first optical sensor module, in particular an interferometric distance sensor, to sense the distance between the distal end of the at least double-clad optical fiber and the tissue to be treated; and/or
2) activate a second optical sensor module, in particular a spectroscopic sensor, to detect one or more spectroscopic properties of the tissue to be treated;
3) and to control the treatment laser source at least in part in dependence of the sensed distance and/or of the detected properties and subject to one or more safety conditions/indicators being fulfilled.

In particular, the control module may configured to receive at least one sensor signal from the first and/or second optical sensor module and to enable output of the treatment laser radiation via at least one optical ports, in particular via the optical output port, only when the at least one sensor signals indicates that a fiber-optic device is coupled to one or more of the optical connectors/optical output ports of the medical treatment device and that the distal end of the optical fiber is positioned at a safe location, e.g. as described in more detail below.

Controlling the treatment laser source at least in part in dependence of the sensed distance and/or of the detected properties may comprise that the control unit selects a laser power and/or another operational parameter of the treatment laser radiation at least in part in dependence of the sensed distance and/or of the detected properties; and controls the treatment laser source to emit treatment laser radiation at the selected laser power and/or the selected other operational parameter. Examples of other operational parameters may include a duty cycle, pulse with, etc. of a pulse train of laser pulses, a duration of emission of the treatment laser radiation, and/or the like. The control unit may be configured to control the treatment laser source at least in part in dependence of the sensed distance and/or of the detected properties so as to control the laser energy deposited at the treatment site.

Preferably, the control unit is configured to repeat acts 1) through 3) while said user input is received or until a termination input is received.

In this respect, the control unit may be configured to automatically control the treatment laser source at least in part in dependence of the sensed distance and/or of the detected properties, or the control unit may be configured to perform said control in an operator-assisted manner. For example, the control unit may be configured to:
i) calculate or otherwise select a laser power and/or another operational parameter of the treatment laser radiation at least in part in dependence of the sensed distance and/or of the detected properties;
ii) display otherwise communicate the calculated or otherwise selected laser power and/or other operational parameter to the operator operating the apparatus and request approval/manual activation of the displayed/communicated values and/or to allow the operator to change and activate the values manually,
iii) control the treatment laser source at least in part in dependence of the operator-approved or operator-changed laser power and/or other operational parameter.

In various embodiments described herein, time-division multiplexing the treatment laser radiation and optical measurements based on sensor laser radiation using the same at least-double clad optical fiber allows the fiber to be very thin while avoiding detrimental interference between the sensing system and the treatment laser, e.g. caused by retro-reflected treatment laser radiation impinging on the detector of the sensor module. In particular, in various embodiments, the medical laser apparatus is configured to time-division multiplex an interferometric distance sensor signal, a spectroscopic sensor signal and a treatment laser radiation for use in surgery, in particular minimally invasive surgery, e.g. ablation and/or coagulation of cancer in the urinary tract, in particular the urinary bladder.

The user input and/or the termination signal may be received via a foot pedal or other suitable user activated input device.

In order to efficiently perform time-division multiplexing, the medical treatment device may be operable to switch the various laser sources sufficiently rapidly, e.g. within 1 ms or faster. To this end, when one or more of the laser sources, e.g. the treatment laser source, is a diode laser, rapid switching may be performed by directly switching of the drive current. When the interferometric distance sensor includes an SLED or a swept source, or when the spectroscopic sensor includes a supercontinuum laser, these may need to be switched in another manner in order to obtain rapid switching, as such lasers may need time to stabilize when switched on. Accordingly, the switching may e.g. be performed by means of an acousto-optic tunable filter (AOTF), a MEMS based device or in another suitable manner. Such an external switch can give rise to reflections. Therefore, when used to switch the sensor laser radiation of the interferometric distance sensor, it may preferably be arranged between the sensor laser source and the coupler/circulator for the receiver. In the spectroscopy engine, the switch may be located on the laser output.

In some embodiments, the control unit is configured to receive information about the disposable fiber-optic device connected to the medical treatment device; and to select the laser power at least in part in dependence of a user-selected laser power, the sensed distance and the received information about the disposable fiber-optic device connected to the medical treatment device.

In some embodiments, the control unit is configured to output a camera control signal operable to control operation of a camera of the endoscope, wherein the control unit is configured to synchronize operation of the camera with the operation of the treatment laser source such that image data is recorded by the camera only when the medical laser apparatus does not emit treatment laser radiation or only emits treatment laser radiation at a substantially reduced intensity, in particular reduced such that the reduced treatment radiation does not unduly affect the camera images. To this end, the treatment laser radiation may be reduced to 5% or less of the intensity of the treatment laser radiation during the preceding treatment cycle, e.g. to 1% or less.

In some embodiments, the control unit is configured to control the laser power of the treatment laser radiation responsive to a detected type of material. To this end, the first optical sensor module may further be configured to detect and distinguish a plurality of different types of material located in front of the distal end of the at least double-clad optical fiber.

FIG. 9 diagrammatically illustrates operation of an example of a medical laser apparatus in accordance with various embodiments disclosed herein, e.g. of the medical laser apparatus described in connection with one or more of the previous figures, in particular an apparatus for treatment of the urinary tract, in particular the urinary bladder. The process is controlled by the control unit of the medical laser apparatus.

Initially, the medical treatment device may be operated in an inactive mode. In the inactive mode, neither emission of the sensor laser radiation nor emission of the treatment laser radiation source are enabled.

Responsive to activation of the medical treatment device by a user input, the medical treatment device may be operated in a safety mode. The activation by the user input may e.g. involve the user pushing a button or selecting a mode of operation. In some embodiments, operation of the device in safety mode may only be enabled when the device has detected that a fiber-optic device has actually been connected to the medical treatment device. In the safety mode, the interferometric distance sensor module of the medical treatment device may be activated and feed sensor laser radiation 4100 through the at least double-clad optical fiber of the disposable fiber-optic device connected to the medical treatment device. The interferometric distance sensor module further receives reflected radiation that is received by the distal end of the at least double-clad optical fiber in response to the emitted sensor laser radiation. The medical treatment device is configured to analyze the received reflected radiation so as to detect whether the distal end is immersed in a liquid, in particular water, saline and/or urine, and/or whether the distal end is positioned close to a surface other than biological tissue. If the distal end is close to a surface other than biological tissue or if the distal end is not immersed in a liquid, in particular water, the control unit may prevent the medical treatment device from emitting treatment laser radiation. Detection of a surface other than biological tissue may indicate that the distal end of the at least double-clad fiber has not been inserted into the endoscope yet or at least has not completely passed through the working channel of the endoscope, or that the endoscope is bent such that the distal end is actually directed towards the outside of the endoscope. Accordingly, preventing the medical treatment device from emitting the treatment laser radiation in this situation prevents unintended damage of the endoscope or other surfaces by the treatment laser radiation.

Failure to detect a liquid, in particular water, may indicate that the distal end of the at least double-clad fiber has not yet been positioned close to the treatment site, in particular not yet been inserted into the urinary bladder. Preventing the medical treatment device from emitting the treatment laser radiation in this situation prevents unintended damage of tissue other than the intended tissue.

Preventing emission of the treatment laser radiation may include preventing the treatment laser source from being powered on and/or preventing a suitable shutter/optical switch from being opened or preventing emission of the treatment laser radiation in another suitable manner.

When, in particular only when, the medical treatment device detects biological tissue in a proximity of the distal end of the at least double-clad optical fiber and water between the distal end and the detected biological tissue, the control unit allows the medical treatment device to exit the safety mode and enter treatment mode.

When operated in treatment mode, output of the treatment laser radiation is enabled, i.e. the operator can initiate emission of the treatment laser radiation by activating a suitable user input device, e.g. a foot pedal or the like. Upon activation of the user input device, the control unit receives an activation signal 4200, e.g. as long as the foot pedal maintains activated. In some embodiments each activation of the foot pedal may cause emission of a single burst of treatment laser radiation (or another predetermined number of bursts), e.g. corresponding to a predetermined—e.g. user selected—amount of laser energy to be delivered to the tissue to be treated (e.g. a predetermined amount of laser energy per unit area).

Responsive to the activation signal, the control unit may repeatedly perform a treatment cycle, e.g. as long as the activation signal 4200 is received or until a termination signal is received, or for a predetermined amount of time, e.g. so as to emit a single burst of laser radiation.

In some embodiments, during each treatment cycle, the control unit initially causes the interferometric distance sensor to emit sensor laser radiation 4110 and to sense the distance between the distal end of the at least double-clad optical fiber and the tissue to be treated. The distance sensor may perform one or more distance measurements during a suitable measurement period, e.g. 10 ms or less. For example, the control unit may compute an average over a plurality of distance measurements. If the interferometric distance sensor detects that the surface in front of the distal end is a surface other than biological tissue or if the distal end is not immersed in a liquid, in particular water, the control unit causes the medical treatment device to exit the treatment mode and return to the safety mode.

Otherwise, upon completion of the distance measurement, the control unit deactivates the sensor laser radiation from the interferometric distance sensor and activates the spectroscopic sensor to emit the spectroscopy sensor laser radiation 4310. The spectroscopic sensor receives light reflected by the tissue to be treated in response to being illuminated with the spectroscopic laser radiation and determines a state/progress of the treatment, e.g. based on an absolute or relative reflected intensity or power in one or more wavelength bands, e.g. at emission peaks of hemoglobin or another suitable molecular marker indicative of the progress of the laser treatment. The spectroscopic measurement may last a suitable measurement period of time, e.g. 10 ms or less. The control unit may then deactivate the sensor radiation from the spectroscopic distance sensor.

Based at least in part on the determined distance and/or on the determined progress, in step 4410 the control unit determines the power and/or duty cycle or other suitable parameter of the treatment laser radiation to be emitted. For example, the control unit may select a smaller power and/or duty cycle if the measured distance is small and a larger power and/or duty cycle if the measured distance is large. The control unit may even prevent emission of the treatment laser radiation if the detected distance is smaller than a safety threshold.

In some embodiments, the selection of the power (or other suitable parameter) of the treatment laser radiation may further be based on a user-selected baseline power (or other suitable parameter). For example, the control unit may automatically adjust the baseline power responsive to the detected distance, e.g. attenuate the laser power relative to the baseline power responsive to a reduced distance.

Alternatively or additionally, the control unit may select the power, duty cycle and/or other suitable parameter of the treatment laser radiation responsive to the detected status/progress of the treatment, e.g. attenuate the laser power and/or duty cycle responsive to an increased progress.

The control unit may further receive information about characteristics of the disposable fiber-optic device currently connected to the medical treatment device, e.g. by reading out an ID tag, e.g. an RFID tag, a QR code or other machine-readable identifier. The control unit may thus select the laser power based on the sensor data from the distance sensor (and, optionally, the sensor data from the spectroscopic sensor), from the pre-set baseline power at the time of device activation and from the received information about the characteristics of the disposable fiber-optic device. The information of the characteristics of the disposable fiber-optic device may e.g. include information about the fiber diameter, numerical aperture and/or the like.

The control unit then controls the treatment laser source to emit the treatment laser radiation 4510 at the selected power. To this end, the control unit may control the treatment laser source to output treatment laser radiation at a suitable output power, e.g. a selected output power in the interval between 10 W and 2200 W or in another suitable interval. In some embodiments the treatment laser radiation may be emitted as a pulse train of treatment laser pulses. The pulses may have a pulse duration of between 1 ms and 10 ms or another suitable pulse duration. The control unit may control the treatment laser to emit the treatment laser radiation for a treatment period which may be predetermined, e.g. as a pre-configured duration or user-selected, or the treatment period may at least partly be automatically selected based on the sensed distance and/or detected progress. In some embodiments, the treatment period may correspond to between 5 and 100 laser pulses, e.g. between 10 and 20 pulses. Upon completion of the treatment period, the control unit deactivates emission of the treatment laser radiation.

Upon completion of the treatment cycle, the control unit again controls the interferometric distance sensor to perform a distance measurement by emitting corresponding sensor laser radiation 4120, followed by a spectroscopic measurement 4320, as described above.

Based at least in part on the determined distance and/or on the determined progress, in step 4420 the control unit determines the power, duty cycle and/or other suitable parameter of the treatment laser radiation to be emitted during the subsequent treatment period. In some embodiments, the determination of the laser power (or other parameter) is not only based on the most recent measurements but further on measurements made during one or more previous treatment cycles. To this end, the control unit may include a memory for storing the results of one or more previous distance measurements and/or status/progress measurements. The results of one or more previous distance measurements may e.g. be used to determine changes in distance due to relative movement of the distal end of the at least double-clad optical fiber and the tissue to be treated. The control unit may thus base the selection of the laser power or other parameter not only on the currently measured distance but also on an observed change of distance, e.g. on rate of change, e.g. whether the distance increases or decreases and, optionally, at what speed. Similarly, the control unit may base the selection of the laser power or other parameter on an observed rate of change of the treatment status.

In some embodiments, the control unit further bases the selected laser power or other parameter on patient feedback received via a suitable user device, e.g. a handheld user device, operated by the patient during the course of the treatment. For example, the patient may activate the user device when the patient experiences pain or discomfort during the treatment. The user device may communicate a corresponding patient feedback signal 4700 to the control unit, which may then adjust the laser power or other parameter for the subsequent treatment cycle responsive to the received feedback.

The control unit then controls the treatment laser source to emit treatment laser radiation 4520 at the selected power and/or other parameter for another treatment period.

The control unit may repeatedly perform treatment cycles as long as the activation signal 4200 is activated or until a termination signal is received or for a predetermined treatment duration, e.g. so as to emit a predetermined burst of laser radiation. As mentioned above, the control unit may also interrupt the treatment responsive to detected unsafe conditions, e.g. that the fiber is directed at a non-tissue surface, that the fiber is no longer immersed in water and/or too close to the tissue to be treated, or when the spectroscopic measurement indicates that the treatment of the tissue has been completed.

It will be appreciated that several modifications may be made to the process for operating the medical laser apparatus. For example, if the medical laser apparatus includes further sensors, measurement periods of these additional sensors may also be included in one or more of the treatment cycles, e.g. in a time-division multiplexed manner as was described above in respect of the distance measurements and spectroscopic measurements. In some embodiments, some or all of the measurements, e.g. the spectroscopic measurement, may not necessarily be performed during each treatment cycle but may e.g. only be performed during a subset of treatment cycles.

While the above embodiments have mainly been described in the context of a fiber-optic device having two separate connectors and a connector/combiner module, other embodiments are also possible, including an embodiment where the treatment radiation and sensor radiation are combined into a single optical fiber inside the medical treatment apparatus.

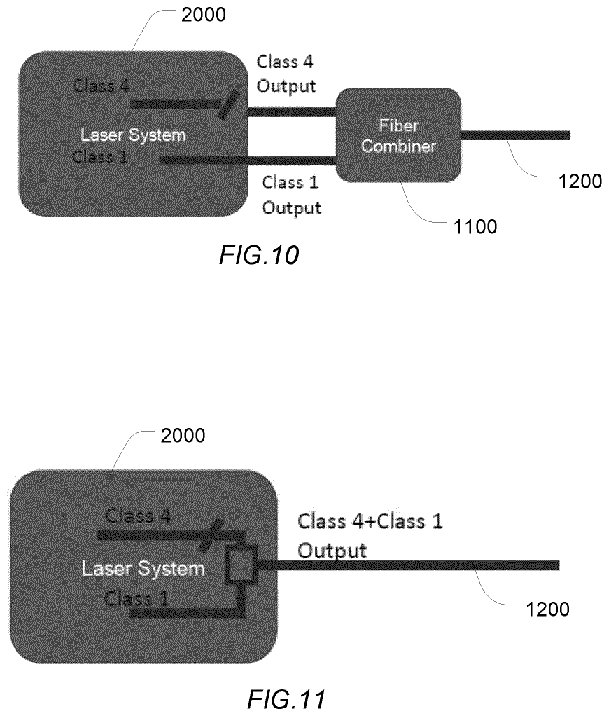

For example, FIG. 10 shows an embodiment where the medical treatment device 2000 has two optical output ports and the outputs from these ports are combined into a single optical fiber 1200 in a separate combiner module 1100. In this embodiment, detection of the presence of water by OCT signals is facilitated by the 2-port system. FIG. 11 shows an example where the outputs from the treatment laser source and the sensor laser source are combined inside the medical treatment device 2000 such that only a single output port is needed. In this embodiment, detection of the insertion into water/into the endoscope/cystoscope may be based on a cystoscope signal (e.g. a modulated LED), by TOF between fiber facets or by a pair of electrodes.

In both embodiments, the sensor radiation is low-power radiation, having a sufficiently small output power to be classified as a class 1 laser according to IEC 60825-1:07-2015, while the treatment laser radiation has an output power so high that it cannot, without special technical safety features, be classified as a class 1 laser according to IEC 60825-1:07-2015 but e.g. as class 4. This means that operators and patients typically have to wear laser goggles and there must normally be interlocks connected to a door switch and a “laser on” lamp outside the room. Accordingly, for the purpose of the present description, the laser sources and optical output ports are also referred to as class 1 and class 4, respectively. However, embodiments of the medical laser apparatus disclosed herein provide protection measures for ensuring that no hazardous emission of radiation can unintentionally be viewed by the person operating the device, the patient, or other person in the vicinity of the apparatus, preferably such that the apparatus can be classified as a class 1C laser according to IEC 60825-1:07-2015. Accordingly, operation of embodiments of the medical laser apparatus disclosed herein requires fewer operational safety precautions, such as goggles, interlocks connected to a door switch, etc. and are thus more user-friendly and efficient in use without compromising safety.

Generally, disclosed herein are embodiments of a medical laser apparatus for performing surgical treatment. The apparatus includes medical treatment apparatus having one or more optical ports. The optical ports include at least one optical output port. The apparatus includes an optical fiber attached to the optical output port for delivering treatment laser radiation into the body of the subject to be treated.

The medical treatment apparatus includes a housing and a treatment laser source accommodated inside the housing. The treatment laser source may be a hazardous laser, in particular a class 4 laser according to IEC 60825-1:07-2015.

The medical treatment apparatus includes safety means that ensure that no hazardous laser radiation is output from the optical output port unless the optical fiber is connected to the optical output port and a distal end of the optical fiber is located inside the body to be treated. Preferably, the safety means are configured such that the medical laser apparatus is safe to use without or at least only minimal personal protective equipment, preferably to a degree that the apparatus can be de-classified to class 1C.

Generally, a safety system may be based on the following procedure:
1) Blocking/disabling the treatment laser (class 4 laser), e.g. by use of a shutter, inside the housing of the laser apparatus.
2) Selectively enable the output of the class 4 laser radiation by:
   i) detecting connection/insertion of a fiber connector to an optical output port of the apparatus
   ii) Turning ON a Class 1 light, optionally subject to the detection of step i)
   iii) Detecting (sufficient) integrity of the connected fiver
   iv) Detecting insertion of the connected fiber into cystoscope and into the bladder by detection immersion of the fiber tip into a liquid (such as water, saline, urine and/or the like) and/or by detecting a valid bladder tissue signal
   v) Opening a shutter or otherwise enable the class 4 laser, subject to conditions i) and iv) having been met

### Stand-Alone Safety System:

In some embodiments, the safety means provide a stand-alone safety system which can operate with conventional endoscopes, such as conventional cystoscopes without requiring specific functionality from the endoscope/cystoscope.

In particular, the apparatus may be configured to emit and detect sensor signals, in particular non-hazardous optical sensor signals for detecting that the distal end of the optical fiber is at a safe position, in particular inside the body to be treated.

To this end, the apparatus may include two optical ports, in particular two optical output ports: one optical output port emits class 4 light, in particular the treatment laser radiation, for treatment; the other optical output port emits and receives class 1 light for sensing.

The class 4 light and the class 1 light can be combined inside the housing of the apparatus and output via a single optical output port, e.g. as illustrated in FIG. 11. Alternatively, the apparatus comprises two optical output ports. In this case, the optical paths of the class 4 light and of the class 1 light may be combined into one fiber outside the apparatus, e.g. by a combiner module as illustrated in FIG. 10.

When a (disposable) fiber is connected to the, or each, optical output port, and when the connection is detected, the apparatus is operated in a safety mode: In this mode, class 1 light is/can be enabled (Class1 refers to laser radiation that is safe under all circumstances). When the apparatus comprises more than one optical output ports, e.g. one for class 4 and one for class 1 light as described above, operation in safety mode may require that a fiber-optic device is connected to each of the optical output ports. In safety mode, the class 1 light is emitted from the fiber tip and reflection signals are collected by the fiber. The class 4 port remains disabled, i.e. output of treatment laser radiation is prevented by electrical means and/or by software means and/or by mechanical means such as by a mechanical shutter in the beam path.

When the reflected class 1 light delivers a signal to the apparatus (as sensed by a suitable sensor of the apparatus) that verifies that the fiber tip is inside the body or in another safe (for people) position, output of the class 4 laser light is/can be enabled.

This verification may involve verification of fulfilment of one or more conditions/indicators, such as the fiber tip being immersed in a liquid such as water, saline and/or urine, and/or the fiber tip receiving light from the endoscope tip and/or the fiber tip being in close proximity to tissue to be treated and/or the like.

It will generally be appreciated that the term enabling output of a laser radiation does not mean that laser radiation is necessarily emitted. Actual emission may further depend on the operator activating the output, e.g. by pressing a foot pedal or the like. Hence enabling output of the laser radiation is intended to mean that the operator may cause the laser radiation to be emitted. When the output is not enabled, the user is prevented from initiating emission of the laser radiation. In other words, enabling output of the treatment laser radiation is intended to refer to enabling a user to control the apparatus to output the treatment laser radiation.

The control process for implementing the safety system may thus include the following:
i) Responsive to detecting insertion/coupling of a fiber connector to the (or to each) optical output port, e.g. using a laser safety interlock system, enabling output of class 1 radiation for transmission to the fiber tip. As class 1 light is non-hazardous, in alternative embodiments emission of class 1 radiation may always be enabled. Optionally detecting (sufficient) integrity of the coupled fiber.
ii) Detecting presence of water at the fiber tip, e.g. by detection a change in reflection, e.g. from about 3.5% (silica/air) to about 0.2% (silica/water) (e.g. corresponding to about a change in reflected power from the fiber tip by a factor of 20, corresponding to about 13 dB detection range. When water is detected, the fiber tip is located in the water-filled channel of the endoscope/cystoscope or in the water filled bladder/urinary tract. Detection can be performed by detection of a change in reflected power or it can be based on a pulsed time-of-flight measurement, where a reflected pulse is present or absent (no water/water presence).
iii) Detecting the light emitted by the cystoscope/endoscope light emitters (e.g. detecting the visible power/spectral attribute of typical cystoscope/endoscope light—alternatively a special cystoscope/endoscope with frequency modulated or otherwise encoded light emission may be employed which allows the apparatus to detect said frequency modulation/encoding to ensure only cystoscopic/endoscopic light is detected—see the integrated solution described below.
iv) Moving the fiber tip forward through the working channel and towards the treatment site, e.g. between 1 mm and 10 mm from tissue to be treated, and detecting the tissue reflection, e.g. using interferometric distance measurements.
v) When condition i) is fulfilled in combination with condition ii) and/or condition iii) and/or condition iv), the class 4 optical output port is enabled (by electrical, software and/or mechanical means)
vi) Disabling the class 4 optical output port:
   If absence of cysto/endoscope light emission (e.g. because the fiber is pulled back, or due to an endoscope failure) is detected by the apparatus,
   if absence of water at the tip (e.g. the fiber is out in air), is detected by the apparatus, or
   if absence of a valid tissue reflection is detected by the apparatus
   if disconnection of the fiber connector is detected or if it is detected that fiber-path integrity is broken, e.g. if the fiber is broken or damage in the optical path has been detected.

The class 1 pathway may preferably be a fiber waveguide with physical contact from the laser source to the fiber tip so as to reduce unwanted reflections in the optical pathway from laser to fiber tip. To this end, the class 1 optical output port may comprise a fiber connector providing physical contact between the respective fiber ends of the fibers being connected by the class 1 optical output port. If the two optical paths are combined, using free-space optics, inside the housing, water detection range may be reduced, e.g. from 13 dB to 3 dB. Also, interferometric/OCT detection of tissue will not be readily feasible. If the two optical pathways, i.e. the treatment and sensor paths are combined, using fiber optics, inside the housing, the connector at the optical output port may be less reliable.

### Endoscope-Integrated Safety System

In some embodiments, the safety means provide an endoscope-integrated safety system that relies on particular functionality of the endoscope/cystoscope it is configured to operate with. In particular, the safety system may rely on the endoscope emitting illumination having one or more attributes specific to the endoscope illumination, in particular light modulated according to a predetermined modulation scheme.

In particular, the apparatus may include two optical ports: one optical output port emits class 4 light for treatment, the other optical port receives and, optionally, emits class 1 light for sensing. As mentioned above, the two optical ports may be separate from another, e.g. as illustrated in FIG. 10, or they may be combined into a single optical port, e.g. as illustrated in FIG. 11.

The Endoscope/cystoscope may be configured to emit modulated/encoded light (e.g. weakly frequency modulated with frequencies that does not affect the endoscope/cystoscope imaging) from the endoscope/cystoscope light emitter(s) that is/are located at the distal end of the endoscope/cystoscope. When a (disposable) fiber is connected to the (or each) optical port and detected, the apparatus will detect light received at the distal end of the connected optical fiber and search for light having the cystoscope modulation code/frequency. When the cystoscope/endoscope modulation is detected, this indicates that the fiber tip is located at the distal end of the cystoscope and the class 4 laser can be enabled.

If the cystoscope/endoscope modulation is suddenly not detected any longer, output of class 4 laser radiation is disabled, e.g. within a suitably short period of time, such as within 100 ms or less.

The control process for implementing the safety system may thus include the following:
i) Detecting insertion/coupling of a fiber connector to the, or each, optical output port, e.g. using a laser safety interlock system,
ii) Detecting the light emitted by the cystoscope/endoscope light emitters having encoded/modulated light emitters
iii) When conditions i) and ii) have been fulfilled, the class 4 optical output port is enabled (by electrical, software and/or mechanical means).
iv) Disabling the class 4 optical output port if absence of the cystoscopic/endoscope light emission is detected by the laser or disconnection of the fiber connector is detected

Optionally, the water detection and/or tissue detection and/or fiber integrity detection described in the context of the stand-alone system may be used as additional condition(s). This may require the apparatus outputting class 1 sensor light, e.g. as described above.

The endoscope-integrated solution may facilitate a two-port implementation inside the housing.

Alternatively or additionally to detect light emitted by the endoscope/cystoscope and received at the distal end of the connected optical fiber, the apparatus may use another mechanism for determining a fiber tip insertion indicator, in particular for detecting whether the distal end of the connected optical fiber extends out of the distal end of the endoscope/cystoscope.

For example, many endoscopes/cystoscopes comprise a camera configured to capture images, e.g. in the form of a video stream, of a target site in front of the distal end of the endoscope/cystoscope. The camera may be implemented by a camera chip located at the distal end of the endoscope or otherwise. The endoscope/cystoscope may be coupled to a display device so as to allow a physician or other person operating the endoscope to view images captured by the camera. The display device may be integrated into the medical laser apparatus disclosed herein or it may be separate therefrom, e.g. by a separate device having its own housing separate from a housing of the medical laser apparatus.

The medical laser apparatus may include a light source configured to emit visible light or otherwise light detectable by the camera of the endoscope/cystoscope. The light source may e.g. a dedicated light source or it may be the pilot light source, or a light source of one of the optical sensors of the apparatus, e.g. the supercontinuum white-light source or other suitable light source of a spectroscopic sensor, or another suitable light source of the apparatus. The apparatus may be configured to emit the camera-detectable light through the connected optical fiber. The apparatus may further be configured to receive a camera signal from the camera, the camera signal being indicative of one or more images captured by the camera while the apparatus emits the camera-detectable light through the connected optical fiber. The apparatus may further be configured to process the received camera signal to detect whether the camera has captured the camera-detectable light. The apparatus may determine that the fiber tip of the connected optical fiber is located at the distal end of the endoscope/cystoscope, or is even protruding out of the distal end of the endoscope/cystoscope, responsive to detecting that the camera has captured the camera-detectable light. Accordingly, the apparatus may enable emission of the treatment laser radiation, in particular class 4 laser radiation, only responsive to detecting that the camera has captured the camera-detectable light. The above process of emitting the camera-detectable light and detecting that the camera of the endoscope/cystoscope has captured the camera-detectable light may be performed prior to enabling emission of the treatment laser radiation and, optionally, continuously or at least intermittently during emission if the treatment laser radiation, e.g. between consecutive pulses of the treatment laser radiation. Failure to detect that the camera has captured the camera-detectable light may thus cause the apparatus to disable emission of the treatment laser radiation.

Preferably, the apparatus is configured to embed a detectable signature into the camera-detectable light, e.g. by pulsing or otherwise modulating the camera-detectable light, e.g. by modulating the intensity or spectral composition of the camera-detectable light or otherwise. The apparatus may thus be configured to detect the embedded signature in the camera signal.

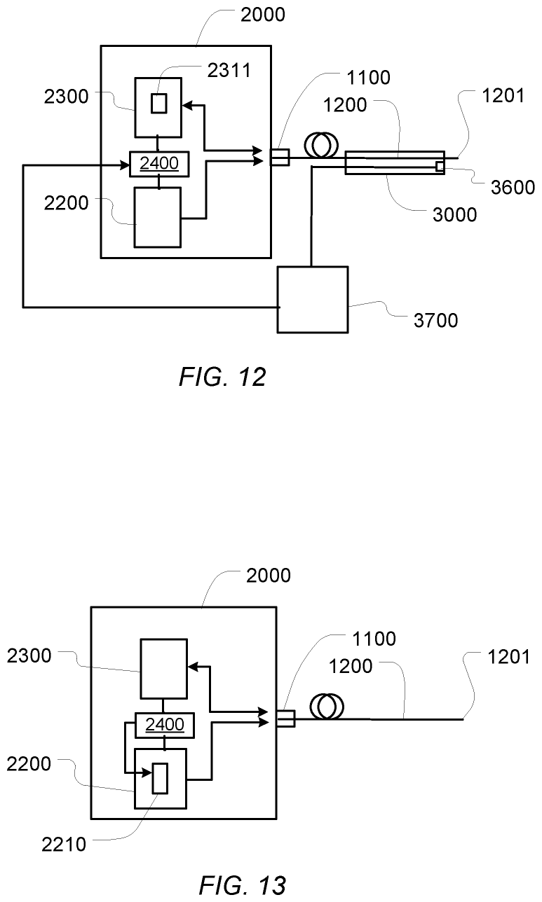

An example of the camera-based detection of a fiber tip insertion indicator will be described with reference to FIG. 12, which diagrammatically illustrates another example of a medical laser apparatus in accordance with embodiments disclosed herein. The apparatus of FIG. 12 is similar to the apparatus of FIGS. 1 and 3 in that it comprises a medical treatment device 2000 and a fiber-optic device, which includes an optical fiber 1200 and a connector module 1100. The medical treatment device 2000 includes a treatment laser source 2200, a control module 2400, and a sensor module 2300, all as described in connection with FIG. 1 and/or with FIG. 3.

The optical fiber 1200 is configured to be inserted and advanced into the working channel of an endoscope 3000, in particular a cystoscope, e.g. as described in connection with FIG. 4, such that the distal end 1201 of the optical fiber 1200 extends out of the working channel of the endoscope. In use, the endoscope is inserted into a vessel or organ of a patient, in particular into the urinary bladder via the urethra. The endoscope 3000 comprises a camera 3600, in particular a camera chip, at its distal end. The camera 3600 provides a camera signal to a display device 3700 coupled to the endoscope 3000 such that the physician can view the images captured by the camera 3600 on the display device 3700.

In the present embodiment, the sensor module 2300 comprises a sensor light source 2311, e.g. an LED, a sensor laser source or the like. The sensor light source 2311 is configured to output visible sensor light through the optical fiber 1200. When the optical fiber is an at least doubled-clad optical fiber, the medical laser apparatus may be configured to output the visible sensor light through a core or through a cladding of the at least doubled-clad optical fiber. It will be appreciated that the sensor light may not need to be visible as long as it is within the spectral range detectable by the camera 3600. Instead of a light source of the sensor module, the medical apparatus may use another light source to emit light that is detectable by the camera 3600, e.g. a pilot light source or a separate light source dedicated for this purpose.

The light source 2311 may be configured and/or be controlled by control module 2400 to emit the camera-detectable light as pulsed light at a suitable predetermined pulse rate detectable by the camera. Alternatively or additionally, the light source 2311 may be configured and/or controlled to modulate the emitted camera-detectable light in a different predetermined manner or to otherwise embed a detectable signature into the emitted camera-detectable light. For example, the emitted camera-detectable light may alternate between two colors or be modulated in a different manner.

The control module 2400 of the medical laser device 2000 is configured to only enable emission of the treatment laser radiation via the optical fiber 1200 when the optical fiber is inserted into the endoscope/cystoscope 3000 with the distal end 1201 of the optical fiber protruding out of the distal end of the endoscope/cystoscope. As described herein, the control module may selectively enable or disable emission of the treatment laser radiation in a variety of ways, e.g. by operating a shutter or by switching the treatment laser source ON/OFF etc.

To this end, the medical laser device 2000 receives the camera signal from the display device 3700, e.g. via an HDMI cable or via another suitable wired or wireless connection capable of forwarding the camera signal. The camera 3600 is thus an example of an external sensor, external to the medical laser device 2000, and the camera signal received from the display device 3700 is an example of a sensor signal received from an external sensor. Nevertheless, it will be appreciated that, in some embodiments, the display device 3700 and the medical laser device may be integrated into the same device, in particular into a single housing. In such and other embodiments, the control module 2400 may receive the camera signal in a different manner, e.g. directly from the camera 3600, via an internal signal path, and/or the like.

The control module 2400 may process the received camera signal so as to detect the embedded signature, e.g. so as to detect a varying light intensity at the predetermined pulse rate, between predetermined colors and/or to detect another predetermined modulation, or otherwise. Responsive to detecting the embedded signature and, optionally responsive to one or more other safety indicators, the control module may enable emission of the treatment laser radiation, e.g. as described herein. Responsive to a failure to detect the embedded signature or, optionally responsive to a failure to detect one or more other safety indicators, the control module may disable emission of the treatment laser radiation, e.g. as described herein.

It will be appreciated that the control module may configured to only enable emission of the treatment laser radiation via the optical fiber 1200 when additional or alternative sensor signals indicate that a fiber-optic device is coupled to the optical output port and that said distal end is positioned at a safe location.

As the medical laser device 2000 controls the emission of the camera-detectable light, including the embedding of a suitable signature into the camera-detectable light, and since the medical laser device 2000 performs the extraction of the embedded signature from the camera signal, this embodiment does not rely on specific properties of the endoscope/cystoscope other than the presence of a camera. In particular, this embodiment does not rely on any detectable properties of light emitted by the endoscope/cystoscope, which may vary greatly between different makes and models of endoscopes/cystoscopes.

Otherwise, the treatment laser source 2200, the control module 2400, the sensor module 2300 and/or the fiber-optic device of the present embodiment may be implemented as described in connection with any of the previous embodiments or otherwise.

According to yet another aspect, disclosed herein are embodiments of a medical laser apparatus, the apparatus comprising:
at least one treatment laser source configured to output treatment laser radiation for treatment of a medical condition,
one or more optical ports configured for coupling the apparatus to a fiber-optic device, the fiber-optic device including at least one optical fiber and having a proximal end and a distal end, the distal end being configured to be advanced through a working channel of an endoscope towards a treatment site of a subject to be treated, the one or more optical ports comprising an optical output port for outputting the treatment laser radiation,
   wherein the treatment laser source is configured to receive a heartbeat signal and to stop output of the treatment laser radiation responsive to a failure to receive the heartbeat signal.

Generally, the heartbeat signal may be indicative of normal operation of one or more components of the apparatus, in particular normal operation of a control module of the apparatus and/or normal operation of at least one sensor of the apparatus. The treatment laser source may be configured to receive the heartbeat signal from the control module and/or directly or indirectly from the at least one sensor.

In some embodiments, the apparatus may comprise a control module that is configured to send a heartbeat signal to the treatment laser source. The control module may be configured to monitor operation of at least some components of the apparatus. For example, the control module may be configured to monitor operation of the at least one sensor, e.g. based on received sensor signals from such sensor and/or based on respective sensor heartbeat signals received by the control module from such sensor. Failure to receive a heartbeat signal by the treatment laser source from the control module may thus indicate faulty operation of the control module and/or of the monitored aspects of the operation of the apparatus. In some embodiments, the control module may be configured to only emit the heartbeat signal when and as long as one or more safety indicators are fulfilled, e.g. one or more of the safety indicators described herein.

In some embodiments, the apparatus comprises at least one sensor that may send a sensor heartbeat signal, e.g. to the control module and/or directly to the treatment laser source. The treatment laser source may be configured to receive the sensor heartbeat signal instead of or in addition to the heartbeat signal from the control module. Accordingly, the control module and/or the treatment laser source may be configured to disable output of the treatment laser radiation if the sensor heartbeat signal is not received, or not received for a predetermined period of time.

The heartbeat signal may be an intermittent signal, in particular a periodic signal emitted at a suitable heartbeat rate, e.g. at a heartbeat rate of between 10 Hz and 100 Hz, or at another suitable update rate. For example, in some embodiments a heartbeat signal may be emitted between every 10 ms and every 20 ms.

The treatment laser source may be configured to emit pulsed treatment laser radiation. In particular, the treatment laser source may be configured to only emit a predetermined number of pulses, e.g. a single pulse or a pulse train of a predetermined length, and to stop emitting further pulses unless a heartbeat signal has been received, within a predetermined period of time. It will be appreciated that the heartbeat rate may be selected in accordance with the predetermined number of pulses and/or the duty cycle of the pulse train. For example, when the treatment laser source is configured to only emit a single pulse of 10 ms, a heartbeat rate of between one heartbeat every 10 ms and every 20 ms may be a suitable choice. It will be appreciated that other embodiments may employ other heartbeat rates and/or other pulse durations.

The treatment laser source may thus comprise a heartbeat monitoring circuit configured to stop the treatment laser source upon failure to receive the heartbeat signal. Preferably the heartbeat monitoring circuit is implemented in hardware, e.g. as a hardware timer which is reset by the heartbeat signal. In some embodiments, the treatment laser source may be configured to only output treatment laser radiation subject to other conditions being fulfilled, such as subject to other signals being received, e.g. subject to receipt of an activation signal indicative of the operator having pressed a foot pedal or other activated the laser treatment.

The control module may be configured to monitor receipt of acceptable signals from the at least one sensor and send its heartbeat signal only subject to receipt of acceptable signals. For example, responsive to the safety indicators being fulfilled and the operator having initiated the treatment, the control module may, optionally subject to receiving the sensor heartbeat signals, control the treatment laser source to emit a preprogrammed pulse sequence. Only subject to the safety features still being fulfilled, optionally only subject to the operator still activating the laser and, optionally, the sensor heartbeat signals still being received, the control module may continue to send its heartbeat signal to cause the treatment laser source to keep emitting another pulse sequence emit a subsequent pulse sequence. The treatment laser source may be configured, preferably implemented by a hardware circuit only, such as by a hardware timer, to automatically switch the treatment laser source off after a preprogrammed sequence. The treatment laser source may further be configured to fire again if a heartbeat signal is received from the control module.

FIG. 13 diagrammatically illustrates an example of a medical laser apparatus in accordance with embodiments disclosed herein, wherein the treatment laser source includes a heartbeat monitoring circuit. The apparatus of FIG. 13 is similar to the apparatus of the previous figures, e.g. of FIG. 1, 3 or 12, in that it comprises a medical treatment device 2000 and a fiber-optic device, which includes an optical fiber 1200 and a connector module 1100. The medical treatment device 2000 includes a treatment laser source 2200, a control module 2400, and a sensor module 2300, all as described in connection with one or more of the previous figures, e.g. FIG. 1, 3 or 12.

The optical fiber 1200 is configured to be inserted and advanced into the working channel of an endoscope (not explicitly shown in FIG. 13), in particular a cystoscope, e.g. as described in connection with FIG. 4, such that the distal end 1201 of the optical fiber 1200 extends out of the working channel of the endoscope. In use, the endoscope is inserted into a vessel or organ of a patient, in particular into the urinary bladder via the urethra.

In the present embodiment, the treatment laser source 2200 includes a heartbeat monitoring circuit 2210 may monitor receipt of the heartbeat signal(s) from the control module and/or the sensor module, and only enable emission of the treatment laser radiation when the heartbeat monitoring circuit receives one or more regular heartbeat signal(s). The heartbeat monitoring circuit may be configured to stop the treatment laser source upon failure to receive the heartbeat signal. In the example of FIG. 13, the heartbeat monitoring circuit receives the heartbeat signal from the control module, which in turn may monitor operation of the sensor module 2300 and/or of other components of the medical laser apparatus. In other embodiments, the heartbeat monitoring circuit may receive heartbeat signals directly from the sensor module and/or from other components; these heartbeat signals may be in additional to or alternative to the heartbeat signals from the control module.

Otherwise, the treatment laser source 2200, the control module 2400, the sensor module 2300 and/or the fiber-optic device of the present embodiment may be implemented as described in connection with any of the previous embodiments or otherwise.

Various aspects have been described with reference to various embodiments. Modifications and alterations will occur to others upon reading the present disclosure. It is intended that the invention be construed as including all such modifications and alterations, including insofar as they come within the scope of the appended claims and the equivalents thereof.

1. A medical laser apparatus, the apparatus comprising:
at least one treatment laser source configured to output treatment laser radiation for treatment of a medical condition,
one or more optical ports configured for coupling the apparatus to a fiber-optic device, the fiber-optic device including at least one optical fiber and having a proximal end and a distal end, the distal end being configured to be advanced through a working channel of an endoscope towards a treatment site of a subject to be treated, the one or more optical ports comprising an optical output port for outputting the treatment laser radiation, and
a control module configured to: receive at least one sensor signal indicative of one or more predetermined safety indicators, the one or more safety indicators including at least: a connection indicator indicative of whether a fiber-optic device is coupled to the optical output port, one or more fiber tip position indicators indicative of whether the distal end of the optical fiber of the coupled fiber-optic device is positioned at a safe location, wherein the control module is further configured to enable output of the treatment laser radiation via the optical output port only when the at least one sensor signal indicates that a fiber-optic device is coupled to the optical output port and that said distal end is positioned at a safe location.

2. A medical laser apparatus according to claim 1, wherein the one or more safety indicators further include one or more fiber integrity indicators and wherein the control module is further configured to enable output of the treatment laser radiation via the optical output port only when the at least one sensor signals indicate that fiber integrity is sufficiently intact.

3. A medical laser apparatus according to claim 1, wherein the one or more fiber tip position indicators include one or more of the following:
a) an insertion indicator indicative of the optical fiber having been inserted into a liquid and/or into the endoscope,
b) an endoscope tip indicator indicative of the optical fiber being positioned at a distal end of a working channel of an endoscope, and
c) a treatment site indicator indicative of the distal end of the optical fiber being in proximity of a potential treatment site.

4. A medical laser apparatus according to claim 3, wherein the control module is configured to only enable the treatment laser radiation to be output when at least two of the insertion indicator, the endoscope tip indicator and the treatment site indicator are fulfilled.

5. A medical laser apparatus according to claim 3, wherein the control module is configured to only enable the treatment laser radiation to be output when at least two of the connection indicator, the fiber integrity indicator and one or more fiber tip position indicators are detected by two separate sensors, respectively, and/or using two separate optical pathways.

6. A medical laser apparatus according to claim 3, wherein the control module is configured to only enable the treatment laser radiation to be output when at least one safety indicator is detected by two separate sensors or by using two separate optical pathways.

7. A medical laser apparatus according to claim 1, wherein the one or more fiber tip position indicators include: an insertion indicator indicative of the optical fiber having been inserted into a liquid, and a treatment site indicator indicative of the distal end of the optical fiber being in proximity of a potential treatment site, and wherein the control module is configured to only enable the treatment laser radiation to be output when at least the insertion indicator and the treatment site indicator are fulfilled.

8. A medical laser apparatus according to claim 1, wherein the control module is configured to only enable the treatment laser radiation to be output when at least two of the safety indicators are fulfilled in a predetermined temporal sequence.

9. A medical laser apparatus according to claim 1, further comprising one or more sensors configured to acquire one or more of the least one sensor signal, and wherein the control module is configured to receive the acquired one or more of the at least one sensor signal from said one or more sensors.

10. A medical laser apparatus according to claim 9, wherein at least one sensor of said one or more sensors is configured to detect the presence of a liquid in front of the distal end of the optical fiber by detection a level of reflection of sensor light by a distal end facet of the optical fiber and/or by a time-of-flight measurement of a reflection of sensor light by the distal end facet and/or by an interferometric measurement.

11. A medical laser apparatus according to claim 9, when dependent on claim 3, wherein at least one sensor of said one or more sensors is configured to detect the at least one endoscope tip indicator by detecting light emitted from a distal end of the endoscope.

12. A medical laser apparatus according to claim 11, wherein the at least one sensor is configured to recognize the light emitted from the distal end of the endoscope by detecting a spectral property of the light or by detection an intensity of the light or by detecting a predetermined modulation and/or encoding of the light.

13. A medical laser apparatus according to claim 9, wherein at least one sensor of the one or more sensors includes an optical sensor configured to receive and detect radiation from at least one of the one or more optical ports.

14. A medical laser apparatus according to claim 13, wherein the optical sensor comprises a sensor radiation source configured to emit sensor radiation.

15. A medical laser apparatus according to claim 14, wherein the sensor radiation has an output power of 5 mW or less.

16. A medical laser apparatus according to claim 13, wherein the optical sensor is configured to perform an interferometric distance measurement or a spectroscopic measurement.

17. A medical laser apparatus according to claim 1, wherein the treatment laser radiation has an optical output power of 10 mW or more.

18. A medical laser apparatus according to claim 1, further comprising a medical treatment device and the fiber-optic device, wherein the medical treatment device comprises at least the treatment laser source, and wherein the fiber-optic device is a disposable fiber-optic device is configured to be detachably and optically coupled to the medical treatment device.

19. A medical laser apparatus according to claim 1, wherein the optical fiber is an at least double-clad optical fiber and wherein the apparatus is configured to output the treatment laser radiation via a cladding of the at least doubled-clad optical fiber.

20. A medical laser apparatus according to claim 19, further comprising an optical side combiner configured to couple the treatment laser radiation into said at least one cladding of the at least double-clad optical fiber.

21. A medical laser apparatus according to claim 1, wherein the treatment laser source is configured to receive a heartbeat signal and to stop output of the treatment laser radiation responsive to a failure to receive the heartbeat signal.

22. A medical laser apparatus according to claim 1, wherein the apparatus is configured to process sensor signals from at least one sensor using two separate signal processing pipelines.

23. A medical laser apparatus according to claim 1, further comprising a light source for emitting light via at least one of the one or more optical ports, the emitted light being detectable by a camera of the endoscope, and wherein the control module is configured to receive a camera signal from the camera, to process the received camera signal so as to detect whether the camera has captured said emitted light, and to enable output of the treatment laser radiation via the optical output port only when the control module has detected that the camera has captured said emitted light.

24. A medical laser apparatus, the apparatus comprising:
at least one treatment laser source configured to output treatment laser radiation for treatment of a medical condition,
one or more optical ports configured for coupling the apparatus to a fiber-optic device, the fiber-optic device including at least one optical fiber and having a proximal end and a distal end, the distal end being configured to be advanced through a working channel of an endoscope towards a treatment site of a subject to be treated, the one or more optical ports comprising an optical output port for outputting the treatment laser radiation,
a light source for emitting light via at least one of the one or more optical ports, the emitted light being detectable by a camera of the endoscope; and
a control module configured to receive a camera signal from the camera, to process the received camera signal so as to detect whether the camera has captured said emitted light, and to enable output of the treatment laser radiation via the optical output port only when the control module has detected that the camera has captured said emitted light.

25. A medical laser apparatus, the apparatus comprising:
at least one treatment laser source configured to output treatment laser radiation for treatment of a medical condition, and
one or more optical ports configured for coupling the apparatus to a fiber-optic device, the fiber-optic device including at least one optical fiber and having a proximal end and a distal end, the distal end being configured to be advanced through a working channel of an endoscope towards a treatment site of a subject to be treated, the one or more optical ports comprising an optical output port for outputting the treatment laser radiation,
wherein the treatment laser source is configured to receive a heartbeat signal and to stop output of the treatment laser radiation responsive to a failure to receive the heartbeat signal.

26. A system comprising:
a medical laser apparatus as defined in any one of the preceding claims, and
an endoscope, in particular a cystoscope, the endoscope having a working channel for receiving the optical fiber, the endoscope further comprising a camera for capturing one or more images of a target site located in front of a distal end of the endoscope.
